SEQUENCE LISTING (7 sequences)

SEQ ID NO: 1 - DNA, 20 bp, Artificial Sequence
  Sense primer for the MNV genome
ctttggaacaatggatgctg

SEQ ID NO: 2 - DNA, 19 bp, Artificial Sequence
  Antisense primer for the MNV genome
cgccatcactcatcctcat

SEQ ID NO: 3 - DNA, 23 bp, Artificial Sequence
  Forward primer (NKP2F)
atgttyagrtggatgagattctc

SEQ ID NO: 4 - DNA, 20 bp, Artificial Sequence
  Reverse primer (NKP2R)
tcgacgccatcttcattcac

SEQ ID NO: 5 - DNA, 18 bp, Artificial Sequence
  NK1PF primer used for GI virus
gcyatgttccgytggatg

SEQ ID NO: 6 - DNA, 21 bp, Artificial Sequence
  NK1PR primer used for GI virus
gtccttagacgccatcatcat

SEQ ID NO: 7 - DNA, 20 bp, Artificial Sequence
  GII.4 Probe (RING2-TP)
tgggagggcgatcgcaatct

(19) United States
(12) Patent Application Publication
(10) Pub. No.: US 2017/0088821 A1
(43) Pub. Date: Mar. 30, 2017

(54) METHODS AND COMPOSITIONS FOR CALICIVIRIDAE

(71) Applicant: UNIVERSITY OF FLORIDA RESEARCH FOUNDATION, INC., GAINESVILLE, FL (US)

(72) Inventors: STEPHANIE KARST, GAINESVILLE, FL (US); MELISSA K. JONES, GAINESVILLE, FL (US)

(21) Appl. No.: 15/310,468

(22) PCT Filed: May 12, 2015
(86) PCT No.: PCT/US15/30361

§ 371 Date: Nov. 11, 2016

Related U.S. Application Data
(60) Provisional application No. 61/992,040, filed on May 12, 2014.

(51) Int. Cl. C12N 7/00; G01N 33/569; C12Q 1/70

(52) U.S. Cl. CPC C12N 7/00; C12Q 1/701; G01N 33/56983; C12N 2770/16051; C12Q 2600/136; G01N 2333/08; G01N 2500/10

(57) ABSTRACT

Disclosed herein are methods and compositions for calicivirus. Methods comprise in vitro cell culture systems used for diagnosis, identification, attenuation and other uses. Compositions comprise in vitro cell culture systems for calicivirus.

### CROSS-REFERENCE TO RELATED APPLICATION

The present application claims the benefit of U.S. Provisional Application Ser. No. 61/992,040, filed May 12, 2014, which is hereby incorporated by reference herein in its entirety, including any figures, tables, or drawings.

### BACKGROUND OF THE INVENTION

This invention relates generally to the field of virology and more particularly to Caliciviridae compositions and methods.

The Caliciviridae family of viruses are members of Class IV of the Baltimore scheme. The genome of these viruses is non-enveloped, non-segmented, positive-sense, single-stranded RNA. Caliciviruses are found in a large number of organisms such as humans, cattle, pigs, cats, chickens, reptiles, dolphins and amphibians.

Caliciviruses are not very well studied because until recently they could not be grown in culture, and there is no suitable animal model. Several research groups are pursuing in vitro culturing of particular caliciviruses, but it is a difficult virus family to study. What is needed are compositions and methods for culturing caliciviruses in vitro and use of in vitro culture methods for diagnosis, virus identification and vaccine development. What is needed are compositions comprising virus-permissive cell cultures.

Noroviruses (NoVs) are nonenveloped plus-strand RNA viruses that cause a majority of gastroenteritis outbreaks worldwide. They are also now recognized as one of the leading causes of severe childhood diarrhea in both industrialized and developing nations (1-3). The cellular tropism of human NoVs, and thus the development of a cultivation system for their in vitro propagation, has long eluded the NoV research community. Based on the enteric nature of NoVs, the most likely target of infection may be the intestinal epithelial cell (IEC). However, IECs have not been demonstrated to be permissive to human NoV (HuNoV) or murine NoV (MNV) infection in vitro (in spite of intensive efforts to infect this cell type with NoVs) although NoV particles can be transcytosed across a polarized epithelium (4-10). Infection of IECs cannot be entirely excluded since viral antigen has been detected in IECs of gnotobiotic pigs and calves infected with a HuNoV (11, 12) and in interferon-deficient mice infected with a MNV (13). The first clue to an alternative NoV cell tropism was provided by the discovery that MNVs productively infect professional antigen presenting cells (APCs) including dendritic cells (DCs) and macrophages (Mφs) (14). There is accumulating evidence that HuNoVs share this cell tropism: HuNoV antigen was detected in lamina propria cells of an intestinal biopsy from an infected volunteer (15); an inactivated HuNoV bound to lamina propria cells, but not IECs, when incubated with human intestinal tissue sections (16); chimpanzees infected with a HuNoV contain infected DCs, but not Mφs or IECs (17); and immunodeficient mice infected with a pool of HuNoVs contain antigen-positive Mφ-like cells in their spleens and livers (18). In spite of this wealth of in vivo supporting data, initial efforts to infect blood-derived monocytes with a HuNoV were unsuccessful (15).

Examples herein demonstrate that MNV strains replicate efficiently in B-cells both in vitro and in vivo. Mature B-cell infection is distinct from Mφ/DC infection-mature B-cells are infected in the absence of significant cytopathic effect and become persistently infected; this is in stark contrast to the rapid and lytic infection of other APCs in culture. Persistent B-cell infection affects the pathogenesis and immune response to NoVs and leads to the development of a successful HuNoV cultivation system, vaccines for Caliciviridae, methods of diagnosing Caliciviridae infections, methods of assaying for antiviral agents that are effective against viruses of Caliciviridae, and other applications that are disclosed herein. Thus, the disclosed invention provides compositions (including virus-permissive cell cultures) and methods for culturing caliciviruses in vitro and use of in vitro culture methods for diagnosis, virus identification and vaccine development.

### BRIEF SUMMARY OF THE INVENTION

Disclosed herein are compositions comprising a calicivirus-permissive cell culture infected with a calicivirus, such as norovirus. Such calicivirus-permissive cell cultures can be comprised of vertebrate cells, in particular B-cells. In some aspects, the vertebrate cells can be murine cells, while in other aspects, the vertebrate cells can be human cells or hybrid cells such as human-mouse fusion cells. In some aspects, a norovirus can be a murine norovirus, while in other aspects, a norovirus can be a human norovirus. Compositions may also comprise other calicivirus-permissive cells.

In an aspect, the present invention comprises methods of replicating a calicivirus, such as a norovirus, in vitro. Methods may comprise inoculating calicivirus-permissive cells with a calicivirus, such as a norovirus, and culturing the cells. In aspects, inoculating calicivirus-permissive cells may comprise infecting the cells with the calicivirus, such as a norovirus. In aspects, the methods can comprise inoculating vertebrate cells such as B-cells. In some aspects, the vertebrate cells can be murine cells, while in other aspects, the vertebrate cells can be human cells or hybrid cells such as human-mouse fusion cells. In some aspects, a calicivirus, such as a norovirus, can be a murine norovirus, while in other aspects, a norovirus can be a human norovirus. Compositions may also comprise other calicivirus-permissive cells.

An aspect disclosed herein comprises methods of detecting calicivirus, such as a norovirus, in a biological or diagnostic sample. For example, a method comprises contacting a cell culture comprising norovirus-permissive cells with a sample, and detecting norovirus viral replication in the cell culture. The sample may be a biological sample, such as a biological sample from a mammal suspected of infection with a calicivirus, such as a norovirus or a diagnostic sample. The mammal can be a human, a laboratory animal such as a rodent, a farm animal, or a companion animal. A biological or diagnostic sample can be a tissue sample, a blood sample, a vomitus sample, a sputum sample, a stool sample or a purified virus. Norovirus-permissive cells may comprise B-cells.

An aspect of the present invention comprises methods of detecting calicivirus, such as a norovirus, in a biological or diagnostic sample comprising performing a cytopathic or non-cytopathic assay, an antibody assay, a nucleic acid detection assay, or a protein detection assay. In an aspect, a cytopathic assay may comprise a dye exclusion assay, an enzyme release assay, a necrosis assay or an apoptosis assay. In an aspect, an antibody assay may comprise a monoclonal or a polyclonal antibody, such as an antibody directed against a norovirus polypeptide and any antigen detection system known in the art, such as a Western blot assay, an ELISA assay, an immunofluorescence assay, an immunoprecipitation assay or a radioimmunoassay. In an aspect, a nucleic acid detection assay may comprise an assay such as a polymerase chain reaction assay or a hybridization assay such as a Northern blot assay.

An aspect of the present invention comprises methods of determining the attachment of a norovirus to permissive target cells. In an aspect, this method may also comprise evaluation of compounds for their ability to inhibit attachment to permissive cells. Such compounds include polyclonal and monoclonal antibodies, anti-virals, biological additives, etc.

An aspect of the present invention comprises methods of identifying a compound having anti-viral activity effective against members of the Caliciviridae family. In an aspect, a method may comprise contacting the compound with a calicivirus-permissive cell culture infected with a calicivirus, such as a norovirus, and detecting inhibition of virus replication. A method may comprise contacting the compound with a calicivirus-permissive cell culture prior to virus infection and assessing for inhibition of infection. Detecting inhibition of viral replication may comprise detecting inhibition of viral nucleic acid synthesis or viral protein synthesis. In an aspect, detecting inhibition of virus replication may comprise performing a plaque assay or a focus forming assay on a calicivirus-permissive cell culture. In an aspect, an assay for identifying anti-viral compounds can be used for identifying compounds having anti-RNA virus activity, anti-single-stranded RNA virus activity, anti-positive strand single-stranded RNA virus activity, anti-positive strand single-stranded RNA activity, no DNA stage virus activity, anti-calicivirus activity, anti-norovirus activity or anti-norovirus attachment activity.

An aspect of the present invention comprises methods of adapting a calicivirus, such as a norovirus, to have a modified host range. In an aspect, a method may comprise serially passaging a norovirus population for three or more generations in calicivirus-permissive cell cultures. Serially passaging may comprise plaque-purifying or focus-forming purifying the calicivirus, such as a norovirus, and growing the plaque-purified or focus-forming purified calicivirus, such as a norovirus, in calicivirus-permissive host cells for three or more serial passages.

An aspect of the present invention comprises methods to test anti-norovirus antibodies for their ability to neutralize viral infection. In an aspect, a method may comprise mixing virus with an antibody and testing whether the antibody reduces infectivity of the virus. Such neutralizing antibodies could be useful vaccine candidates.

An aspect of the present invention comprises methods to generate attenuated viruses as vaccine candidates. In an aspect, a method may comprise passaging the virus through permissive cells numerous times such that the virus evolves to replicate efficiently in cell culture but acquires mutations in virulence genes that are needed during in vivo infections, thus producing an attenuated virus which may be used as a vaccine.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIGS. 1A, B, and C are graphs and protein gels showing murine cell lines infected with MNV. (FIG. 1A) These graphs indicate virus growth curves of RAW 264.7 cells (macrophage cell line; known to be permissive, a positive control), CMT-93 (intestinal epithelial cell line; negative control), M12 (mature B-cell line), and WEHI (immature B-cell line). Cell lines were infected with either MNV-1 (1) or MNV-3 (2) at MOI 5. (FIG. 1B) These graphs indicate viability assays of mock infection (1), MNV-1 infected (2), and MNV-3 infected (3) RAW 264.7 cells, M12, and WEHI cell lines. Cells were infected with either MNV-1 or MNV-3 at MOI 5. FIG. 1C shows protein gels of viral protein synthesis of RAW 264.7 cells (1), M12 (2), and WEHI (3) cell lines. Cells were infected with either MNV-1 or MNV-3 at MOI 5.

FIGS. 2A, B and C are photomicrographs and a graph showing the infectivity of MNVs in various cell types. FIG. 2A shows RAW 264.7, 24 hpi, and FIG. 2B shows M12 cells, 96 hpi, were infected with either MNV-1 or MNV-3 at MOI 5. FIG. 2C is a graph showing the percentage of virally infected B-cells in M12, MOI 20, (1) and WEHI, MOI 5, (2) cell lines.

FIGS. 3A, B, C and D are graphs, photomicrographs and a Western blot showing that MNVs persistently infect mature B-cells (M12 cells). (FIG. 3A) The virus titers in the supernatants were determined using a standard TCID₅₀ assay. (FIG. 3B) Infectivity was analyzed by IFA using anti-ProPol antibody and DAPI. FIG. 3C are photomicrographs of the viral ProPol signal (red) and the DAPI staining of nuclei (blue) shown from passage 10 (P10) cultures in mock, MNV-1, and MNV-3 infected cells. FIG. 3D is a representative Western blot of cell lysates from persistently MNV-1- or MNV-3-infected M12 cultures.

FIGS. 4A, B and C are graphs showing that B-cells support MNV infection in vivo. Groups of C57BL/6 mice (black bars) and B-cell^{-/-} mice (white bars) were infected with 10⁷ TCID₅₀ units (FIG. 4A) MNV-1 virus titers or (FIG. 4B) MNV-3 virus titers and harvested at 0.5 dpi (n=10) or 1 dpi (n=10). Virus titers were determined in the indicated tissues by standard plaque assay. (FIG. 4C) Peyer's patch B-cells were purified from mock-inoculated or MNV-infected wild-type mice (n=8) at 1 dpi and analyzed for viral genome using qRT-PCR; bulk Peyer's patch cells were analyzed in parallel.

FIGS. 5A, B, C and D are graphs, a Western blot, and photomicrographs showing human norovirus infection of human B-cells. (FIG. 5A) A GII.4-Sydney human norovirus (HuNoV)-positive stool sample was applied to the human B-cell line BJAB either unfiltered (black line) or filtered through a 0.2 micron membrane (gray line). At 0, 3, and 5 dpi, the cells and supernatant from an entire well were harvested and analyzed for viral genome by qRT-PCR. These data represent averaged values from 13 independent experiments. (FIG. 5B) The same experiment was performed as described in FIG. 5A except unfiltered (black bars) and filtered (gray bars) inocula were either untreated (solid bars) or treated with UV light which is expected to inactivate live virus (hatched bars). The fold-increase in genomes between 0 and 3 or 5 dpi is indicated. (FIG. 5C) BJAB cells were inoculated with a mock inoculum or unfiltered GII.4-positive stool. At 0, 3 and 5 dpi, cell lysates were generated for the purpose of Western blotting with an anti-NS6 antibody. (FIG. 5D) BJAB cells were inoculated with the unfiltered GII.4-positive stool sample and analyzed by IFA using anti-capsid antibody and DAPI. FIG. 5D are photomicrographs of the viral capsid signal (red) and the DAPI staining of nuclei (blue) at 0 and 5 dpi.

FIG. 6 is a graph showing cell line comparison of growth of GII.4-Sydney infection. Direct infections were performed on a panel of B-cell and macrophage lines indicated on the x-axis using unfiltered GII.4-positive stool. Samples were collected at 0 and 4 dpi for each cell line and viral genomes quantified by qRT-PCR. The fold-increase in genomes over this time period is displayed in the graphs.

FIGS. 7A and B represent the co-culture infections described herein. (FIG. 7A) The diagram is a schematic showing a co-culture in vitro cell culture system in a transwell. An example of intestinal epithelial cells (IECs) is the HT-29 cell line. Examples of B-cells are the BJAB cell line. (FIG. 7B) The graph indicates human norovirus infection in a transwell system described herein comprising B-cells located on the basolateral side of an epithelial layer. Unfiltered (black bar) or filtered (gray bar) GII.4-positive stool was inoculated into the apical supernatant of a transwell in vitro culture system. At 0 and 3 dpi, the lower chamber containing BJAB cells was collected and analyzed for viral genomes by qRT-PCR. A control well that did not get seeded with B-cells in the basal chamber was inoculated with unfiltered stool (white bar); the lack of genome replication in this control proves that virus is not simply diffusing across the confluent epithelial barrier. The data are reported as fold-increase in genomes between 0 and 3 dpi.

FIGS. 8A and B are graphs demonstrating that B-cell infection is enhanced by HBGA-like carbohydrate expressing bacteria. (FIG. 8A) Filtered GII.4-positive stool was premixed with increasing CFU of heat-killed HBGA-positive E. cloacae, 10⁶ CFU heat-killed HBGA-negative E. coli, LPS, or synthetic H-type HBGA (H) before inoculating BJAB cells. As controls, unfiltered (UF) and filtered (F) stool were used in parallel and unfiltered stool pre-incubated with an anti-VP1 antibody (α-VP1) was used as an additional negative control. All conditions were then inoculated onto permissive BJAB cells and genome increases between 0 and 3 dpi determined. (FIG. 8B) Unfiltered (UF; black bar) or filtered (F; white bar) GII.4-positive stool, or F pre-incubated with 10⁶ CFU heat-killed E. cloacae (F+10⁶; blue bar) were applied to the apical supernatant of the co-culture system. Viral genome increases in the basal compartment were determined at 3 dpi.

FIG. 9 is a graph demonstrating infection by MNV-1 and MNV-3 in the presence or absence of bacteria. Groups of C57BL/6 mice were administered a cocktail of four antibiotics for five consecutive days by oral gavage, and then in their drinking water for the duration of the experiment (Abx; red bars). Control mice received PBS in the same manner (gray bars). Both groups of mice were infected with 10⁷ TCID₅₀ units MNV-1 (left panel) or MNV-3 (right panel). At 1 dpi, virus was titered from the distal ileum (DI), mesenteric lymph nodes (MLNs), and colon using a standard virus plaque assay. The asterisks indicate statistical differences.

FIGS. 10A and B represent the viral attachment assay we have developed for a human norovirus. (FIG. 10A) Unfiltered (black bars), filtered (gray bars), or filtered GII.4-positive stool pre-incubated with synthetic H-type HBGA (filtered+H; white bars) were incubated with BJAB cells at 4° C. for 10, 30, or 60 minutes. Unattached virus was then removed from cells by washing and the percent of viral genomes that remained associated with cells was determined. The data are reported as percent attachment. (FIG. 10B) Filtered GII.4-positive stool (F) was pre-incubated with increasing concentrations of pig gastric mucin (PGM) and then tested in the attachment assay described above. Unfiltered stool (UF) was included as a positive control.

FIG. 11 is a graph indicating use of the human B-cell infection system to test for antibody neutralizing activity. Unfiltered GII.4-positive stool was pre-incubated with increasing concentrations of a commercially available polyclonal anti-VP1 antibody (concentrations indicated on a log-scale on the x-axis) and then tested in direct B-cell infections. The fold-increase in viral genomes between 0 and 3 dpi is indicated on the y-axis. The black dashed line indicates the genome increase of unfiltered stool without antibody as a positive control; the gray dashed line indicates complete neutralization, or no increase in viral genome numbers.

### DETAILED DESCRIPTION

The present invention comprises compositions, methods and kits comprising members of the Caliciviridae family. Compositions comprise in vitro cell culture and attachment systems for members of the Caliciviridae family comprising B-cells, and methods for making and using such in vitro culture and attachment systems. Methods of the present invention comprise continuous culture or attachment of members of the Caliciviridae family, diagnostic methods, screening tests, detection of diseases or conditions associated with human or other animal infection by members of the Caliciviridae family, kits for accomplishing such methods or comprising compositions of the present invention, development of vaccines in the form of attenuated live viruses, and testing of virus-specific antibodies for neutralization capacity or passive immunity.

Members of the Caliciviridae family include, but are not limited to, lagovirus, norovirus, sapovirus, vesivirus, recovirus and valovirus genera, from which species have been identified, such as Nebovirus, Norwalk virus, Sapporo virus, Vesicular exanthema of swine virus, rabbit hemorrhagic disease virus, and other Caliciviridae viruses such as St. Valerien-like viruses, chicken calicivirus and newly identified caliciviruses. Vesiviruses and certain sapoviruses (porcine) can already be cultured in vitro in feline and porcine kidney cells, respectively. Rabbit hemorrhagic disease virus is non-cultivable, similar to human noroviruses prior to the present invention. A new cluster of noroviruses has been proposed which has a number of canine caliciviruses. Additional proposed Caliciviridae genera include Bavovirus (chickens), Nacovirus (turkeys and chickens), and Secalivirus (found in sewage). These caliciviruses are contemplated by the present invention and though the disclosure is directed to noroviruses for clarity, this is not to be seen as limiting. As used herein, the term “norovirus” can refer to unmodified, wild-type norovirus, e.g., norovirus obtained from an individual with viral gastroenteritis, or may be an identified, purified, or laboratory standard norovirus. A host range-modified norovirus refers to norovirus modified, with regard to its host range, using well known viral laboratory methods, e.g., norovirus grown in vitro for multiple passages that grows in cells other than the original cells in which the virus grew.

In Vitro Cell Culture

Aspects of the present invention comprise an in vitro cell culture system for a calicivirus, such as norovirus. Methods and compositions for culturing norovirus are described herein. The methods and compositions described herein use well known laboratory techniques which can be found in laboratory manuals such as Sambrook, J., et al., Molecular Cloning: A Laboratory Manual, 3rd ed., Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; and Harlow, E., Using Antibodies: A Laboratory Manual, Cold Spring Harbor Laboratory Press, Cold Spring Harbor, N.Y., 1999.

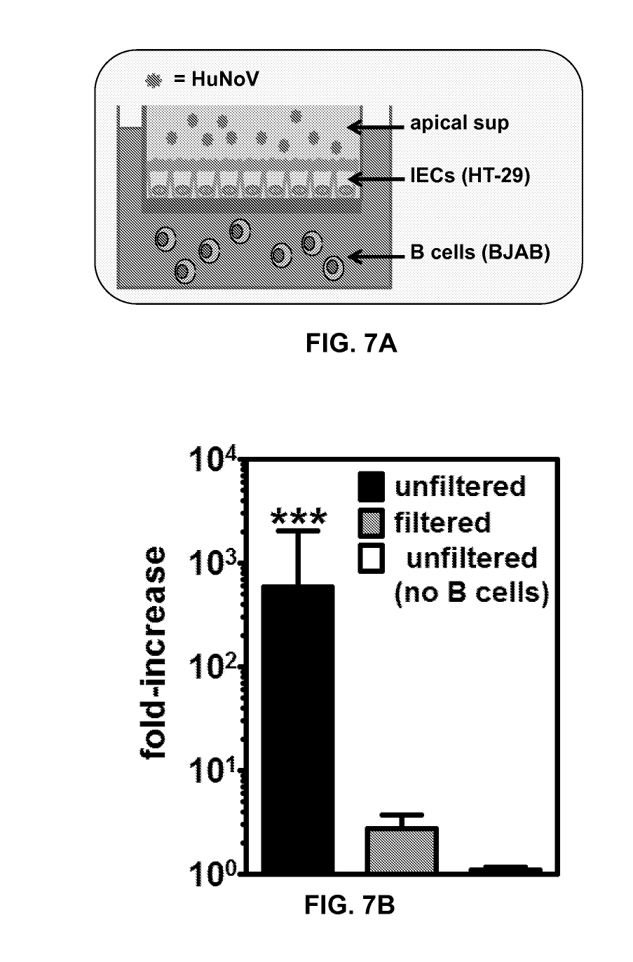

An in vitro cell culture system for human norovirus is disclosed herein and comprises norovirus-permissive host cells, which are human B-cells. A cell culture system for murine norovirus is disclosed herein and comprises norovirus-permissive host cells, which are murine B-cells. As used herein, a “norovirus-permissive cell” is a cell in which a norovirus replicates following infection with live norovirus. Both murine and human in vitro cell culture systems for calicivirus, such as norovirus, may comprise B-cells in an in vitro cell culture system, or an in vitro cell culture system may comprise a co-culture of B-cells and intestinal epithelial cells in a transwell in vitro cell culture system. See FIG. 7A, which is a graphic representation of an in vitro cell culture system comprising a transwell culture vessel comprising a co-culture of cells comprising intestinal epithelial cells and B-cells. An in vitro cell culture system for norovirus may comprise a transwell culture vessel comprising intestinal epithelial cells grown on a transwell membrane and B-cells in the lower chamber, wherein the B-cells are located on the basal side of the intestinal epithelial cells. An in vitro cell culture system disclosed herein may comprise a transwell in vitro cell culture system. An in vitro cell culture system for norovirus may comprise a norovirus-permissive cell infected with a norovirus, wherein the norovirus-permissive cell is a B-cell, and the norovirus-permissive cell may be infected with a norovirus. In an aspect, the cells can be murine cells, human cells, or other cells. Cells in the in vitro culture system can be, for example, B-cells or a co-culture of B-cells and intestinal epithelial cells as disclosed herein. In an aspect, a calicivirus can be a norovirus that is a murine norovirus or a human norovirus, such as a Norwalk virus.

As disclosed herein, an in vitro cell culture system may further comprise HBGA-like carbohydrates or one or more sources of HBGA-like carbohydrates. The HBGA-like carbohydrates may be added directly to the in vitro cell culture system. The HBGA-like carbohydrates may be mixed with a biological or diagnostic sample and the resulting mixture added to the in vitro cell culture system. The HBGA-like carbohydrates may be mixed with a filtered biological or diagnostic sample and the resulting mixture added to the in vitro cell culture system. The HBGA-like carbohydrates may be mixed with a purified virus sample and the resulting mixture added to the in vitro cell culture system.

As used herein, a “B-cell” or B-lymphocyte is a type of lymphocyte in the humoral immunity of the adaptive immune system. B-cells can be distinguished from other lymphocytes, such as T cells and natural killer cells (NK cells), by the presence of a protein on the B-cell's outer surface known as a B-cell receptor (BCR). This specialized receptor protein allows a B-cell to bind to a specific antigen. In birds, B-cells mature in the bursa of Fabricius. In mammals, immature B-cells are formed in the bone marrow. In vivo, the principal functions of B-cells are to make antibodies against antigens, to perform the role of antigen-presenting cells (APCs), and to develop into memory B-cells after activation by antigen interaction. A B-cell is not in the macrophage or dendritic cell lineage of hematopoietic system cells.

As used herein, “norovirus replication” can be understood to include various stages in norovirus life cycle, such as, for example, binding or attachment of a norovirus to a host cell, entry into the host cell, trafficking, processing, genome release, translation, transcription, assembly, and release. Norovirus replication can also be detected by measuring norovirus protein activity, for example polyprotein protease activity, viral RNA polymerase activity, VPG activity and NTPase activity. For example, measurement of an increased accumulation of viral RNA or viral protein in infected cells can be considered an indication of viral replication, although an increase in virus particle production is not measured. Any of the aspects of norovirus replication and reproduction may be used, for example, in a test of an anti-viral agent, anti-viral activity can be detected by detecting inhibition of a norovirus protein activity, such as inhibition of polyprotein protease activity, viral RNA polymerase activity, VPG activity or NTPase activity, or by detecting inhibition of a norovirus protein accumulation, such as inhibition of polyprotein protease accumulation, viral RNA polymerase accumulation, VPG accumulation or NTPase accumulation.

Aspects of the present invention comprise use of an in vitro cell culture system for a calicivirus, such as norovirus, in methods such as diagnostic methods, development of assays for detection or quantification of viral attachment or replication, selection of mutant viruses with desirable properties, identification of mutant viruses, screening of potential anti-viral compounds, and development of vaccines. In an aspect, the vertebrate cells can be murine cells, human cells, or other cells. Cells in the in vitro culture can be, for example, B-cells or a co-culture of B-cells and intestinal epithelial cells as disclosed herein. In an aspect, a calicivirus can be a norovirus that is a murine norovirus or a human norovirus, such as a Norwalk virus.

An in vitro cell culture system for a calicivirus, such as norovirus, may comprise bacteria comprising carbohydrates that mimic human histo-blood group antigens (HBGAs) or one or more biological sources for HBGAs, isolated HBGA-like carbohydrates or porcine gastric mucin. As used herein, HBGA-like carbohydrates include bacteria expressing HBGA-like carbohydrates, other biological sources of HBGA-like carbohydrates, such as human cells, or isolated HBGA-like carbohydrates (isolated from a biological source such a cell or a bacteria), or synthetic HBGA-like carbohydrates made by synthetic methods in a laboratory, or porcine gastric mucin. HBGA-like carbohydrates may be used herein interchangeably with bacteria that express HBGA-like carbohydrates or other natural or synthetic sources of HBGAs such as porcine gastric mucin, and those of skill in the art are able to determine the meaning from such use.

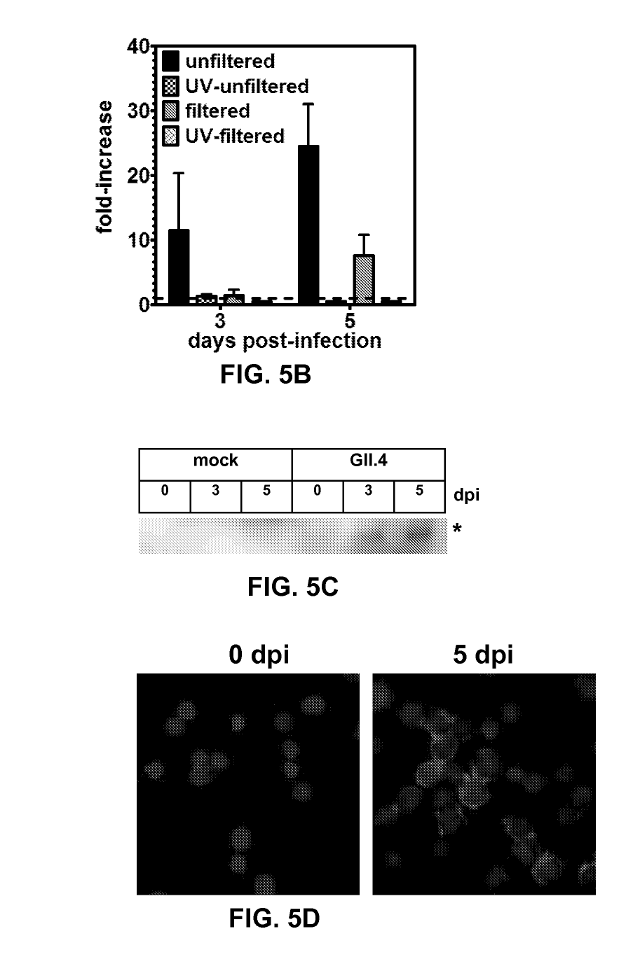

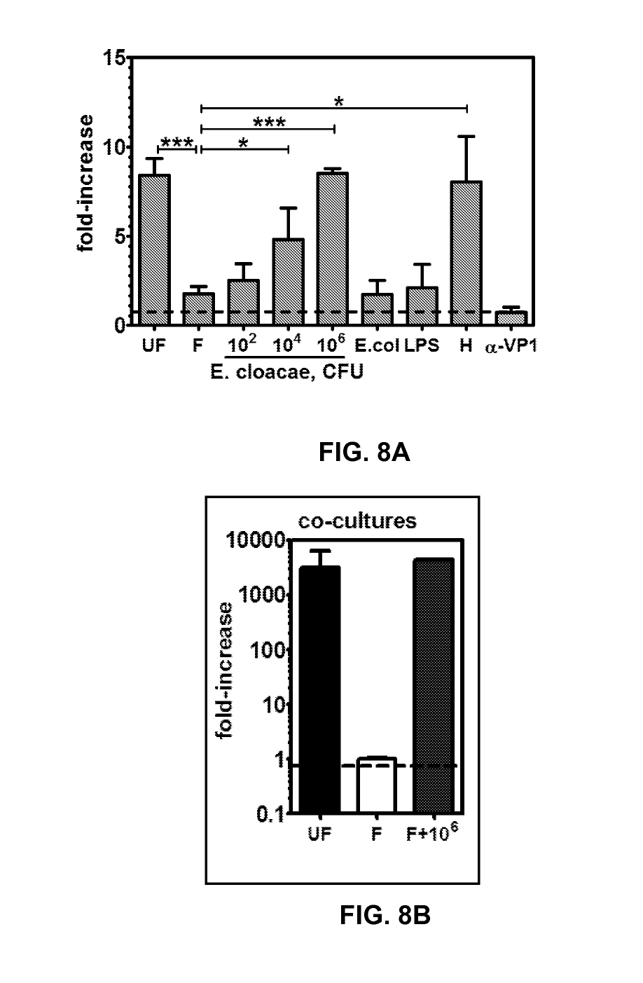

As shown herein, human noroviruses can infect and replicate in human B-cells when using virus-positive stool samples as a source of virus. When the stool sample was filtered through a 0.2 micron filter, which removes bacteria in the stool but not small viruses like noroviruses, infectivity was ablated (see FIGS. 5A and 7B). When a specific bacteria, Enterobacter cloacae (E. cloacae), was premixed with the filtered stool, infectivity was restored (see FIGS. 8A and 8B). Furthermore, filtration of stool samples ablated viral attachment to the target cell but the addition of HBGA (including but not limited to synthetic

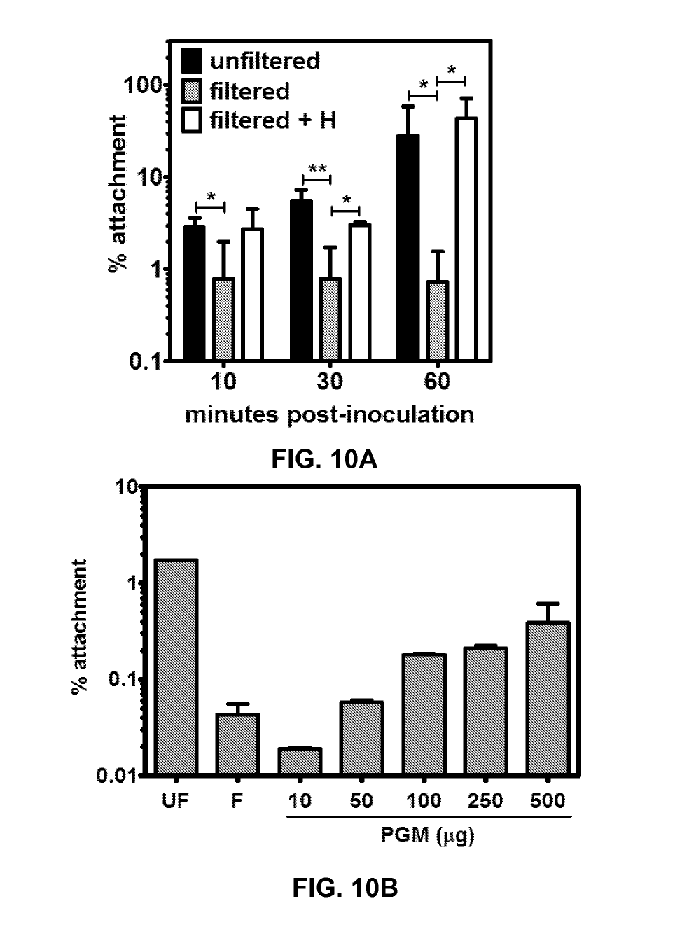

HBGA, HBGA-expressing bacteria and HBGA containing porcine gastric mucin) restores attachment of norovirus to the target cell (see FIGS. 10A and 10B). Though not wishing to be bound by any particular theory, it is currently believed that human noroviruses require HBGA-like carbohydrates for infectivity, such as bacteria comprising carbohydrates that mimic human histo-blood group antigens (HBGAs) or another source for HBGAs for infection of B-cells. Incubating B-cells with bacteria comprising carbohydrates that mimic human histo-blood group antigens (HBGAs) (referred to herein as HBGA-like carbohydrates) or another source for HBGAs prior to infection aids in infectivity by norovirus. Bacteria comprising carbohydrates that mimic human histo-blood group antigens (HBGAs) or another source for HBGAs are effective in human norovirus infection in the in vitro cell culture systems disclosed herein such as a B-cell only in vitro cell culture system or a transwell in vitro cell culture system, also referred to herein as a co-culture system. Incubation of HuNoVs with bacteria expressing the appropriate HBGA-like molecule, or other sources of HBGAs including synthetic HBGA-like carbohydrates or host sources of HBGAs such as bodily secretions, including but not limited to saliva, will facilitate HuNoV infection of B-cells in direct infections and in the transwell in vitro cell culture system (co-culture system). Purified virus that has been concentrated over a sucrose cushion and fractionated on a cesium chloride gradient (standard purification technique for noroviruses) will infect B-cells in the in vitro cell culture systems disclosed herein when premixed with a source of HBGA-like carbohydrates.

It is known that bacteria, including certain commensal bacteria, express carbohydrates that are indistinguishable from human HBGAs (22-25). Though not wishing to be bound by any particular theory, from the findings herein, it is currently believed that human noroviruses can bind these HBGA-like carbohydrates on commensal bacteria in the gut lumen and this is used for viral infection of B-cells. It is theorized that any bacteria expressing the correct HBGA-like carbohydrate for a given human norovirus strain will facilitate viral infection. The types of bacteria known to express HBGA-like carbohydrates are diverse and include Enterobacter, Escherichia, Shigella, and Helicobacter. There are likely many other commensal bacteria (e.g. strict anaerobes) that express HBGA-like molecules.

It is known that people generate antibodies directed to non-self HBGAs (isoantibodies that mediate blood transfusion reactions). Though not wishing to be bound by any particular theory, it is currently believed that these antibodies are secreted into the gut lumen where the antibodies bind and exclude commensal bacteria expressing non-matched carbohydrates. So a person's HBGA expression profile will shape his or her microbiota through exclusion of bacteria expressing non-matched HBGA-like carbohydrates. Though not wishing to be bound by any particular theory, it is currently believed that the correlation between a person's HBGA genetics and his or her susceptibility to specific human norovirus strains may be due to HBGA expression.

Methods of the present invention comprising an in vitro cell culture system may further comprise adding HBGA-like carbohydrates or one or more sources of HBGA-like carbohydrates. The HBGA-like carbohydrates may be added directly to an in vitro cell culture system. The HBGA-like carbohydrates may be mixed with a biological or diagnostic sample and the resulting mixture added to the in vitro cell culture system. The HBGA-like carbohydrates may be mixed with a filtered biological or diagnostic sample and the resulting mixture added to the in vitro cell culture system. The HBGA-like carbohydrates may be mixed with a purified virus sample and the resulting mixture added to the in vitro cell culture system.

A method comprises determining the susceptibility of a subject to norovirus infection comprising identifying the HBGA of the subject and testing a norovirus for its binding to that HBGA. If the virus binds the HBGA of the subject, the subject is susceptible to that norovirus strain. In an aspect, a method comprises obtaining from a subject a sample comprising bacteria or HBGA-like carbohydrates, such as a fecal sample, adding the sample to an ELISA assay described herein to determine the type of HBGA present in the sample, and determining the norovirus susceptibility of the subject.

Replicating a Virus in vitro

In an aspect, the present invention comprises methods of replicating a norovirus in vitro. A method may comprise inoculating norovirus-permissive cells with a norovirus, and culturing the cells. In an aspect, the cells may be human cells and the norovirus is a human norovirus. Inoculating norovirus-permissive cells may comprise infecting the cells with a norovirus. In an aspect, a method may comprise inoculating vertebrate cells, which can be B-cells or co-cultures of B-cells and intestinal epithelial cells. In an aspect, the vertebrate cells can be murine cells, human cells, or other cells. Cells in the in vitro culture can be, for example, B-cells or a co-culture of B-cells and intestinal epithelial cells as disclosed herein. In an aspect, a calicivirus can be a norovirus that is a murine norovirus or a human norovirus, such as a Norwalk virus.

Continuous Culture of Norovirus

Aspects of the present invention comprise a norovirus-permissive cell culture infected with a norovirus. A norovirus permissive cell culture can be maintained using routine cell culturing techniques well known to skilled artisans. A norovirus-permissive cell culture may comprise vertebrate cells, such as B-cells, or a co-culture of B-cells and intestinal epithelial cells. In an aspect, the vertebrate cells can be murine cells, human cells, or other cells. Cells in the in vitro culture can be, for example, B-cells or a co-culture of B-cells and intestinal epithelial cells as disclosed herein. In an aspect, a calicivirus can be a norovirus that is a murine norovirus or a human norovirus, such as a Norwalk virus.

In an aspect, a method for continuous culture of norovirus comprises a) contacting a norovirus to cells in a transwell in vitro cell culture system comprising a transwell culture vessel comprising intestinal epithelial cells grown on a transwell membrane and B-cells in the lower chamber, wherein the B-cells are located on the basal side of the intestinal epithelial cells; b) allowing the norovirus to replicate; and c) passaging the norovirus produced in the culture vessel to a subsequent in vitro culture vessel comprising a transwell culture vessel comprising intestinal epithelial cells grown on a transwell membrane and B-cells in the lower chamber, wherein the B-cells are located on the basal side of the intestinal epithelial cells. Passaging the norovirus produced or the norovirus infected cells may be repeated one or more times. In an aspect, the cells can be murine cells, human cells, or other cells. Cells in the in vitro culture can be, for example, B-cells or a co-culture of B-cells and intestinal epithelial cells as disclosed herein. In an aspect, a calicivirus can be a norovirus that is a murine norovirus or a human norovirus, such as a Norwalk virus.

In an aspect, a method for continuous culture of norovirus comprises a) contacting a norovirus to B-cells; b) allowing the norovirus to replicate; and c) passaging the norovirus produced in the culture vessel to a subsequent in vitro culture vessel comprising B-cells. Passaging the norovirus produced or the norovirus infected cells may be repeated one or more times. In an aspect, the cells can be murine cells, human cells, or other cells. Cells in the in vitro culture can be, for example, B-cells or a co-culture of B-cells and intestinal epithelial cells as disclosed herein. In an aspect, a calicivirus can be a norovirus that is a murine norovirus or a human norovirus, such as a Norwalk virus.

Method for Norovirus Detection

In an aspect, the invention comprises methods of detecting a member of the Caliciviridae family, such as a norovirus, in a biological or diagnostic sample. A method may comprise contacting an in vitro cell culture comprising norovirus-permissive cells, such as B-cells, with the sample, and detecting norovirus viral replication or attachment in the cell culture. A biological sample may be a diagnostic sample, such as a diagnostic sample from an animal suspected of infection with a member of the Caliciviridae family, such as a norovirus. The animal may be a mammal, for example, a human, a laboratory animal such as a rodent, for example a mouse, a rat, or a guinea pig, a farm animal such as a rabbit, cow or a sheep, or a companion animal such as a cat or dog. A biological/diagnostic sample may also be drinking water or a food item such as fresh fruits and vegetables, raw or processed seafoods or agents such as irrigation waters, or additives used to harvest or produce a food item. A biological/diagnostic sample may also be a tissue sample, a blood sample, vomitus, or a stool sample. A tissue sample can be from any tissue or body fluid (blood sample, vomitus, stool sample, saliva, sputum, etc.) that is suspected of infection with a member of the Caliciviridae family, such as a norovirus, such as, for example, liver, kidney, brain, blood, or saliva. In an aspect, the permissive cells can be murine cells, human cells, or other cells. Cells in the in vitro culture can be, for example, B-cells or a co-culture of B-cells and intestinal epithelial cells as disclosed herein. In an aspect, a calicivirus can be a norovirus that is a murine norovirus or a human norovirus, such as a Norwalk virus.

In an aspect, a method for calicivirus detection comprises a) contacting a biological or diagnostic sample (e.g., a diagnostic sample from a subject) or a biological/diagnostic sample pre-mixed with an HBGA-like or HBGA containing compound to an in vitro cell culture system comprising B-cells as disclosed herein, or to an in vitro cell culture system comprising a transwell culture vessel comprising intestinal epithelial cells grown on a transwell membrane and B-cells in the lower chamber, wherein the B-cells are located on the basal side of the intestinal epithelial cells; and b) after a predetermined time, testing the supernatant and/or the B-cells for calicivirus replication.

In an aspect, a method for norovirus detection comprises a) contacting a biological or diagnostic sample from a subject or a biological or diagnostic sample pre-mixed with an HBGA-like or HBGA containing compound to an in vitro cell culture system comprising B-cells as disclosed herein, or to an in vitro cell culture system comprising a transwell culture vessel comprising intestinal epithelial cells grown on a transwell membrane and B-cells in the lower chamber, wherein the B-cells are located on the basal side of the intestinal epithelial cells; and b) after a predetermined time, testing the supernatant and/or the B-cells for norovirus replication.

In an aspect, further steps of a method for calicivirus, such as a norovirus, detection, comprise a) identifying the calicivirus, such as a norovirus, found in an in vitro cell culture system. Identifying may comprise genetically identifying the virus found. The sample may be obtained from a subject suspected of infection with a member of the Caliciviridae family, such as a norovirus. The subject may be for example, a human, a rodent, a cow, a pig, a rabbit, a cat, a dog, a chicken, a reptile, a dolphin or an amphibian. A sample may be a stool sample, a vomitus sample, a tissue sample, blood sample or food item (as discussed above). Detection may be by any means known to those of skill in the art, including, but not limited to, those disclosed herein, such as a virus detection assay such as a cytopathic assay, an antibody assay, a nucleic acid detection assay or a protein detection assay. A cytopathic assay may be a dye exclusion assay, an enzyme release assay or an apoptosis assay. An antibody assay may comprise a Western blot assay, an ELISA assay, an immunofluorescence assay, an immunoprecipitation assay or a radioimmunoassay. A nucleic acid detection assay can be a polymerase chain reaction (PCR) assay or a hybridization assay. In an aspect, a method of detecting a member of the Caliciviridae family, such as a norovirus, in a biological or diagnostic sample can comprise detecting a host cell change that results from viral infection. An in vitro cell culture system as disclosed herein, comprising B-cells, may be used for detecting a host cell change in the B-cells. A host cell change can be, for example, a change in morphology, molecular composition, or cytopathicity. For example, a method for detecting a member of the Caliciviridae family, such as a norovirus, in a biological or diagnostic sample can comprise performing a cytopathic assay, an antibody assay, a protein detection assay or a nucleic acid detection assay. A cytopathic assay can be, in some configurations, a dye exclusion assay, an enzyme release assay, a necrosis assay, or an apoptosis assay. A dye exclusion assay can be, in non-limiting example, a trypan blue exclusion assay, or a fluorescent dye exclusion assay such as a propidium iodide exclusion assay. In some configurations, an antibody assay can use a monoclonal or a polyclonal antibody, such as a monoclonal antibody directed against a viral polypeptide. Any antigen detection system known in the art, such as a Western blot assay, an ELISA assay, an immunofluorescence assay, an immunoprecipitation assay or a radioimmunoassay, can be used to detect the presence and/or quantity of a norovirus. A protein detection assay may comprise, in non-limiting example, a gel electrophoresis assay, a column chromatography assay, and an enzyme assay. A nucleic acid detection assay can be an assay such as a polymerase chain reaction assay or a hybridization assay such as a Northern blot assay, or others known to those of skill in the art.

In an aspect, a method of using an in vitro cell culture system disclosed herein comprises a method of detecting the presence of a disease or condition due to norovirus in a subject comprising obtaining a tissue or body fluid sample from a subject, and contacting an in vitro cell culture vessel disclosed herein, for example, an in vitro culture system comprising B-cells. In an aspect, a method comprises contacting an in vitro cell culture system comprising a transwell culture vessel comprising intestinal epithelial cells grown on a transwell membrane and B-cells in the lower chamber, wherein the B-cells plus HBGA-containing compound or bacteria are located on the basal side of the intestinal epithelial cells and wherein the tissue or body fluid sample from the subject contacts the intestinal epithelial cells or the supernatant of the well comprising the intestinal epithelial cells. The presence of a disease or condition is indicated by detection of norovirus replication in the B-cells.

In an aspect, a method of using an in vitro culture system disclosed herein comprises a method for diagnosing calicivirus, such as a norovirus, in a subject having symptoms associated with calicivirus, such as a norovirus, infection comprising assaying a biological or diagnostic sample from a subject having symptoms associated with calicivirus, such as a norovirus, infection for the presence of calicivirus, such as a norovirus. In an aspect, a method comprises contacting the biological or diagnostic sample with or without HBGA containing compounds or bacteria with an in vitro cell culture system comprising B-cells as disclosed herein. In an aspect, a method comprises contacting the biological or diagnostic sample with an in vitro cell culture system comprising a transwell culture vessel comprising intestinal epithelial cells grown on a transwell membrane and B-cells mixed with HBGA containing compounds or bacteria in the lower chamber, wherein the B-cells are located on the basal side of the intestinal epithelial cells and wherein the tissue or body fluid sample from the subject contacts the intestinal epithelial cells or the supernatant of the well comprising the intestinal epithelial cells. Diagnosis of the viral infection comprises detection of the calicivirus such as norovirus comprises identifying the virus by methods known to those of skill in the art.

In an aspect, a method of using an in vitro culture system disclosed herein comprises assaying a biological or diagnostic sample for the presence of a calicivirus such as norovirus comprising assaying a biological or diagnostic sample, with or without HBGA containing compounds or bacteria mixed therewith, from a subject for the presence of a calicivirus, such as a norovirus. In an aspect, a method comprises contacting the biological or diagnostic sample with an in vitro cell culture system comprising B-cells as disclosed herein. In an aspect, a method comprises contacting the biological or diagnostic sample with an in vitro cell culture system comprising a transwell culture vessel comprising intestinal epithelial cells grown on a transwell membrane and B-cells in the lower chamber, wherein the B-cells are located on the basal side of the intestinal epithelial cells and wherein the tissue or body fluid sample from the subject contacts the intestinal epithelial cells or the supernatant of the well comprising the intestinal epithelial cells. The biological/diagnostic sample can be mixed with or without HBGA containing compounds or bacteria prior to contacting the biological/diagnostic sample with the cell culture system. Detection of the norovirus comprises identifying the norovirus by methods known to those of skill in the art. In an aspect, the cells can be murine cells, human cells, or other cells. Cells in the in vitro culture can be, for example, B-cells or a co-culture of B-cells and intestinal epithelial cells as disclosed herein. In an aspect, a calicivirus can be a norovirus that is a murine norovirus or a human norovirus, such as a Norwalk virus.

In an aspect, a method of using an in vitro culture system disclosed herein comprises a method for testing the ability of antibodies generated in a calicivirus infection, such as antibodies to the norovirus particle, to neutralize infectivity or attachment of a norovirus or other calicivirus. Without an in vitro cell culture cell culture system, such as those disclosed herein, currently used tests rely on a surrogate assay which tests whether an antibody inhibits binding to a putative attachment receptor as a measure of virus neutralization. However, there is no evidence that inhibition of binding to this receptor correlates with neutralization of infection. The culture systems disclosed herein allow for direct measurement of antibody activity. For example, a method for testing antibodies for neutralization of a norovirus comprises contacting a particular type of norovirus with antibodies isolated from a subject infected with that type of norovirus to affect binding of the antibody to the norovirus to form an antibody/norovirus complex. In an aspect, a method comprises contacting the antibody/norovirus complex with an in vitro cell culture system comprising B-cells as disclosed herein. In an aspect, a method comprises contacting the antibody/norovirus complex with an in vitro cell culture system comprising a transwell culture vessel comprising intestinal epithelial cells in the upper chamber and B-cells in the lower chamber, wherein the B-cells are located on the basal side of the intestinal epithelial cells and wherein the antibody/norovirus complex contacts the intestinal epithelial cells or the supernatant of the well comprising the intestinal epithelial cells. A further step comprises detection of the norovirus attachment to, or infection of, the B-cells. If the virus cannot bind to the B-cells, or the B-cells are not infected, the antibody was a neutralizing antibody and if the B-cells are infected with norovirus, the antibody was not a neutralizing antibody. In an aspect, the cells can be murine cells, human cells, or other cells. Cells in the in vitro culture can be, for example, B-cells or a co-culture of B-cells and intestinal epithelial cells as disclosed herein. In an aspect, a calicivirus can be a norovirus that is a murine norovirus or a human norovirus, such as a Norwalk virus.

A Kit

In an aspect, the present invention comprises kits for performing methods disclosed herein or kits comprising norovirus-permissive cells, such as B-cells. In an aspect, a kit comprising an in vitro cell culture system comprising B-cells as disclosed herein. In an aspect, a kit comprises an in vitro cell culture vessel comprising a transwell in vitro cell culture system comprising intestinal epithelial cells grown on a transwell membrane and B-cells in the lower chamber, wherein the B-cells are located on the basal side of the intestinal epithelial cells. In an aspect, a kit comprising an in vitro attachment system comprising B-cells as disclosed herein. A kit may further comprise instructions, media, other cells, positive and negative controls, and other components known to those of skill in the art. In an aspect, the cells can be murine cells, human cells, or other cells. Cells in the in vitro culture can be, for example, B-cells or a co-culture of B-cells and intestinal epithelial cells as disclosed herein. In an aspect, a calicivirus can be a norovirus that is a murine norovirus or a human norovirus, such as a Norwalk virus.

Detecting Anti-Viral Compounds

In an aspect, the present invention comprises methods of identifying a compound having anti-viral activity against a virus of the Caliciviridae family. “Anti-viral activity,” as used herein, may comprise inhibiting viral activity at any stage in a virus' life cycle. Anti-viral activity may also comprise inhibiting binding of a virus to a cell or inhibiting cellular uptake of the virus. Anti-viral activity may comprise, in non-limiting example, inhibition of viral replication, inhibition of viral gene expression, or inhibition of a viral protein accumulation or activity Inhibition of a viral protein accumulation or activity can comprise, in non-limiting example, inhibition of a member of the Caliciviridae family, such as a norovirus, polyprotein protease accumulation, inhibition of norovirus RNA polymerase accumulation, inhibition of VPG accumulation, inhibition of NTPase accumulation, inhibition of norovirus polyprotein protease activity, inhibition of norovirus RNA polymerase activity, inhibition of VPG activity, or inhibition of NTPase activity. Standard methods well known in the art for measuring or detecting viral protein accumulation or activity can be used, for example, enzyme assays and antibody assays.

In an aspect, a method for identifying a compound having anti-viral activity may comprise contacting a candidate potential anti-viral compound with a calicivirus-permissive cell culture infected with a member of the Caliciviridae family, such as a norovirus, and detecting inhibition of viral replication or attachment to B-cells. For example, a method may comprise contacting a candidate potential anti-viral compound with a norovirus-permissive cell culture infected with a norovirus, and detecting inhibition of norovirus replication. In certain aspects, a candidate anti-viral compound can be added to a calicivirus infected culture, such as an infected norovirus-permissive culture, at a concentration of from about 1 picomolar to about 100 millimolar, or from about 1 nanomolar to about 100 micromolar. Detecting inhibition of viral replication in some embodiments may comprise detecting inhibition of viral nucleic acid synthesis or viral protein synthesis. In an aspect, detecting inhibition of viral replication may comprise performing a plaque assay on the cell culture. A plaque assay may comprise determining a titer of virus accumulated in a plaque formed by infected cells in the presence of the candidate anti-viral molecule. A focus forming assay may comprise determining a titer of virus accumulated in the presence of the candidate anti-viral molecule. In these configurations, assays for identifying anti-viral compounds can be used for identifying compounds inhibiting viral attachment to B-cells or having anti-RNA virus activity, anti-single-stranded RNA virus activity, anti-positive strand single-stranded RNA virus activity, anti-positive strand single-stranded RNA activity, no DNA stage virus activity, anti-calicivirus activity, or anti-norovirus activity. In an aspect, anti-viral activity can be detected by detecting differences between infected cells contacted with a candidate anti-viral agent and control (not infected) cells. Such differences can comprise, in non-limiting example, gene expression differences, antigenic differences, enzyme activity differences, dye-staining differences, or morphological differences (as revealed by light microscopy or electron microscopy). In an aspect, anti-viral activity can be detected by performing a cytopathic effects (CPE) inhibition assay in which the anti-viral activity reduces or prevents norovirus-induced CPE. In an aspect, the cells can be murine cells, human cells, or other cells. Cells in the in vitro culture can be, for example, B-cells or a co-culture of B-cells and intestinal epithelial cells as disclosed herein. In an aspect, a calicivirus can be a norovirus that is a murine norovirus or a human norovirus, such as a Norwalk virus.

In an aspect, a method for screening for an active agent to inhibit norovirus attachment, replication or infection comprises a) contacting a candidate active agent (compound) with the cells or the supernatant of an in vitro culture system comprising B-cells, as disclosed herein, or an in vitro cell culture vessel comprising a transwell cell culture system comprising intestinal epithelial cells grown on a transwell membrane and B-cells in the lower chamber, wherein the B-cells are located on the basal side of the intestinal epithelial cells; b) adding a calicivirus, such as a norovirus, to the in vitro culture system; c) allowing the norovirus to replicate; and d) detecting the inhibition of virus replication in the supernatant of the B-cells compared to virus replication in the supernatant of B-cells not contacted with the candidate active agent and/or detecting the attachment of virus to the B-cells compared to virus attachment to B-cells not contacted with the candidate active agent. In an aspect, the cells can be murine cells, human cells, or other cells. Cells in the in vitro culture can be, for example, B-cells or a co-culture of B-cells and intestinal epithelial cells as disclosed herein. In an aspect, a calicivirus can be a norovirus that is a murine norovirus or a human norovirus, such as a Norwalk virus.

In an aspect, the present invention may comprise methods of adapting norovirus to have a modified host range. For example, a method may comprise serially passaging a norovirus population for three or more generations in norovirus-permissive cell cultures. Serially passaging may comprise plaque-purifying a norovirus and growing the plaque-purified norovirus in norovirus-permissive host cells for two serial passages, three serial passages, or more serial passages. Serially passaging may comprise performing a focus forming assay to identify infected cells by immunostaining instead of cytopathic effect; the norovirus purified in this way will be grown in norovirus-permissive host cells for two serial passages, three serial passages, or more serial passages. In an aspect, adapting the host range-modified norovirus to growth in a vaccine production-approved cell line can comprise infecting the approved cell line with host range-modified norovirus, and growing the virus. Methods for producing a vaccine against a virus using a virus exhibiting reduced virulence through serial passage adaptation (Sabin, A. B., Ann. N.Y. Acad. Sci. 61: 924-938, 1955) or through genetic engineering (e.g., by altering codons) are well known to skilled artisans. In an aspect, an in vitro cell culture system may comprise B-cells as disclosed herein. In an aspect, an in vitro cell culture vessel may comprise a transwell in vitro cell culture system comprising intestinal epithelial cells grown on a transwell membrane and B-cells in the lower chamber, wherein the B-cells are located on the basal side of the intestinal epithelial cells. Cells in the in vitro culture can be, for example, B-cells or a co-culture of B-cells and intestinal epithelial cells as disclosed herein. In an aspect, a calicivirus can be a norovirus that is a murine norovirus or a human norovirus, such as a Norwalk virus.

Reduced Virulence Virus/Vaccine

In an aspect, the present invention may comprise a method for preparing a reduced virulence norovirus, comprising a) passaging norovirus in an in vitro cell culture system disclosed herein; and b) selecting a reduced virulence norovirus, wherein the virulence is reduced compared to wild-type or non-adapted norovirus. In an aspect, an in vitro cell culture system may comprise B-cells as disclosed herein. In an aspect, an in vitro cell culture vessel may comprise a transwell culture vessel comprising intestinal epithelial cells grown on a transwell membrane and B-cells in the lower chamber, wherein the B-cells are located on the basal side of the intestinal epithelial cells. Cells in the in vitro culture can be, for example, B-cells or a co-culture of B-cells and intestinal epithelial cells as disclosed herein. In an aspect, a calicivirus can be a norovirus that is a murine norovirus or a human norovirus, such as a Norwalk virus. In an aspect, the present invention comprises a norovirus vaccine comprising a therapeutically effective amount of a reduced virulence norovirus.

The invention can be further understood by reference to the examples which follow.

### EXAMPLES

### Example 1

### Materials and Methods

Cell lines. The M12 (26), WEHI-231 (27), and BJAB (28) B-cell lines were cultured in RPMI with 10% fetal bovine serum; M12 and WEHI-231 media was supplemented with 50 μM β-mercaptoethanol. RAW264.7, CMT-93, 293T, and HT-29 cell lines were cultured in DMEM with 10% fetal bovine serum. Media for all experiments contained 100 U/mL penicillin and 100 μg/mL streptomycin. For co-culturing human IECs and B-cells, HT-29 cells were grown to confluency on hanging wells. Polarization was confirmed prior to infections using fluorescein-conjugated dextran 3000 Da (FITC-dextran) exclusion. Specifically, 4 μg/mL FITC-dextran was loaded into the apical chamber. At 45 min. post-incubation, basal supernatants were collected and analyzed for fluorescent signal using a Synergy BioTek plate reader. Basal supernatants with fluorescence maxima less than 10% of the signal detected in a control well with no HT-29 cells were considered to be polarized. BJAB-cells were then cultured in the basal chamber.

Viruses. Recombinant MNV-1 and MNV-3 that have been previously described (19) were used for all experiments. In brief, virus stocks were generated by transfecting 5 μg infectious clone per 10⁶ 293T cells, freeze-thawing cells after 1 d, and infecting RAW264.7 cells with 293T lysates at MOI 0.05. RAW264.7 lysates were generated when>90% of cells displayed CPE and were purified through a 25% sucrose cushion. Titers of all MNV stocks were determined using a standard TCID₅₀ assay (29). For HuNoV infections, a stool sample that tested positive for the GII.4-Sydney strain was provided by the Centers for Disease Control (CDC); this stool sample tested negative for GI and GIV HuNoVs, sapovirus, astrovirus, rotavirus, and enteric adenovirus. We also performed pilot studies with two de-identified stool samples testing positive for the GII.4-Sydney strain that were obtained from Shands Hospital associated with the University of Florida. Stool samples were not processed prior to inoculation of cells unless otherwise indicated. Titers of HuNoV stocks were determined by quantitative RT-PCR (RT-qPCR) using established genogroup-specific primer sets (30) (see below for details).

MNV infections and growth curves. Cells were infected at MOI 5 unless otherwise indicated and incubated for 1 h on ice. Cells were then washed to remove unbound virus and incubated at 37° C. At the indicated times post-infection, culture supernatants were collected and virus titers determined using a standard TCID₅₀ assay (29).

Cell viability assays. Cells were incubated with propidium iodide (BD Pharmingen) at a final concentration of 2.5 μg/mL for 5 min. at room temperature. Flow cytometric analysis was performed on a FACSCalibur instrument (BD Biosciences) and the CellQuest Pro software was used to analyze data. Data are reported as the percentage of cells that did not incorporate dye (% viable cells).

Western blotting. Cells were lysed in lx Laemmli sample buffer, and proteins were separated by SDS-PAGE and transferred to polyvinylidene fluoride (PVDF) membranes. Previously described antibodies to the MNV-1 protease-polymerase (ProPol) nonstructural proteins (31), the VP1 major capsid protein (32), and the VP2 minor capsid protein (19) as well as a mouse anti-actin antibody (EMD Millipore) were used sequentially to probe the PVDF membranes for MNV studies. The blots were stripped using Restore PLUS Western Blot Stripping Buffer (Thermo Scientific) in between antibody incubations. A previously described antibody to a genogroup II HuNoV NS6 peptide (18) was used to probe membranes for HuNoV studies.

MNV immunofluorescence assay. Cells were fixed in 4% paraformaldehyde in phosphate buffered saline (PBS) and deposited onto glass slides using a Cytopro Cytocentrifuge (Wescor Inc.). Cells were then permeabilized with 0.1% Triton X-100/4% PFA in PBS and stained with anti-ProPol antibody (31) at a 1:1000 dilution followed by an Alexa Fluor 594-conjugated secondary antibody (Invitrogen). Antibodies were diluted in 1% bovine serum albumin in PBS. Nuclei were stained with mounting media containing DAPI (Vector Laboratories, Inc.). Stained cells were imaged with a Zeiss Axioplan-2 Upright Fluorescent Microscope. To determine infectivity frequencies, three random 40X fields were counted per sample and the data averaged.

MNV persistence assays. M12 cells were infected at MOI 5 and plated at 3×10⁵ cells/mL. At 2 dpi, cells were washed extensively to remove extracellular virus and re-plated at 3×10⁵ cells/mL. This procedure was repeated every 2 d. The culture supernatant and cells were collected at every fifth passage for viral detection using the described TCID₅₀ assay and IFA, respectively.

Mice, virus infections, and in vivo tissue titer determination. Wild-type B6 mice (Jackson #00064) and Ighm^{™/Cgn} mice (also known as μMT, Jackson #002288), which lack B-cells, were bred and housed in animal facilities at the University of Florida under specific-pathogen-free conditions. 129S6/SvEv Stat1^{™/Rds} (Taconic #2045; referred to as Stat1^{-/-}) mice were bred and housed in animal facilities at the University of Michigan under specific-pathogen-free conditions. Six- to ten-week old, sex-matched mice were used in all experiments. Virus infections were performed perorally (p.o.) with 25 μL inoculum, unless otherwise indicated. For neutral red-labeled MNV-1 infections, virus was administered through oral gavage with 100 μL inoculum and infections were carried out in the dark. For determining in vivo tissue titers, specified tissues were dissected from perfused mice, weighed, and homogenized in media by bead beating using 1.0 mm zirconia/silica beads (BioSpec Products, Inc.). Plaque assays of tissue samples have been described (14, 33). For titering tissues from mice inoculated with neutral red-labeled MNV-1, tissues were homogenized in the dark. One portion of each homogenate was titered in the dark (total titer) while a second portion was exposed to light for 30 min. and titered in a parallel plaque assay (light-insensitive titer representing replicated virus).

Magnetic separation of cells. Groups of B6 mice (n=8) were inoculated p.o. with mock inoculum or 10⁷ TCID₅₀ units MNV-1 or MNV-3. At 1 dpi, Peyer's patches were dissected and pooled single cell suspensions were prepared by grinding through a 70-μm strainer. B-cells were positively selected from bulk Peyer's patch cells using the EasySep Mouse CD19 Positive Selection Kit and EasySep magnet (STEMCELL Technologies Inc.). The purity of separated B-cells was measured by flow cytometric analysis and determined to be >97% in each experiment.

MNV quantitative RT-PCR. RNA was extracted from purified Peyer's patch cells using the E.Z.N.A MicroElute Total RNA Kit (Omega Bio-Tek Inc.) and cDNA was synthesized using the ImProm-II Reverse Transcription System (Promega). Quantitative PCR was performed on each cDNA sample in triplicate. Primers for the MNV genome were sense 5′-CTTTGGAACAATGGATGCTG-3′ (SEQ ID NO: 1) and antisense 5′-CGCCATCACTCATCCTCAT-3′(SEQ ID NO: 2). Serial dilutions of a plasmid with the viral target sequence were used to generate a standard curve for the purpose of determining viral genome copy numbers in each sample.

Antibiotic depletion of the murine intestinal microbiota. The intestinal microbiota was depleted by oral gavage of a standard antibiotic cocktail, as previously described (34, 35). In brief, mice were orally gavaged daily for five days with 10 mg each of vancomycin (Fisher Scientific), ampicillin (Acros Organics), metronidazole (Acros Organics), and neomycin (Sigma). After the fifth day of gavage, antibiotics were added to the drinking water at a concentration of 1 g/L for ampicillin, metronidazole, and neomycin and 500 mg/L for vancomycin. Fresh fecal samples were collected from mice after the fifth day of oral gavage, homogenized, plated on brain-heart infusion (BHI) agar plates with 10% sheep blood, and incubated under anaerobic conditions at 37° C. for 2 d followed by aerobic conditions at 37° C. for 1 d to confirm efficient microbial depletion. Mice were maintained on the antibiotic- or PBS-containing water for the duration of the experiment, and infections were only performed after antibiotic-treated mice were verified to be free of detectable culturable bacteria.

HuNoV infections and genome copy number determination. The indicated numbers of viral genome copy numbers were applied directly to BJAB cells; or in the apical chamber of a hanging well containing polarized HT-29 cells grown on the membrane and BJAB cells in the lower chamber. Cultures were incubated at 37° C. for the indicated times, at which point the contents of the entire well (direct infections) or the entire basal chamber contents (co-culture) were freeze/thawed two times and the cell lysate used for RNA extraction and RT-qPCR detection of viral genome copy number. Published primers NK2PF (5′-ATGTTYAGRTGGATGAGATTCTC) (SEQ ID NO: 3) and NK2PR (5′-TCGACGCCATCTTCATTCAC) (SEQ ID NO: 4) were used to amplify genogroup II viruses at a final concentration of 200 nM each (30). Two pl of undiluted cDNA was added to each reaction and each sample was run in triplicate. Samples were amplified on a iCycler iQ (Bio-Rad) with SYBR Green Master Mix (Thermo Scientific) under the following cycling conditions: 95° C. for 10 min. followed by 41 cycles of 95° C. for 15s, 58° C. for 30 s, and 72° C. for 30 s. Serial dilutions of a plasmid with the viral target sequence provided by Park et al. (30) were used to generate a standard curve for the purpose of determining viral genome copy numbers in each sample. In certain experiments, aliquots of HuNoV stocks were inactivated with 200,000 μJ cm⁻² UV for 25 min. prior to inoculation; aliquots of HuNoV stocks were incubated with 10 μg/mL anti-VP1 antibody (Abcam, catalog # ab92976) for 1 h at 37° C. prior to inoculation of BJAB cells to test for antibody-mediated virus neutralization; and stocks were filtered through a 0.2 μm membrane prior to inoculation.

HuNoV immunofluorescence assay. The protocol described for MNV IFAS was used with the following modifications. After permeabilization, cells were blocked with 5% normal goat serum. A Tyramide Signal Amplification Kit (Invitrogen) was then used according to manufacturer's protocol. Briefly, cells were stained with anti-VP1 mouse monoclonal antibody (Abcam; catalog #ab80022) at a 1:25 dilution followed by anti-mouse IgG-HRP and Alexa Fluor 594-conjugated tyramide.

HuNoV passaging studies. A passage 0 (P0) virus stock was prepared by infecting BJAB cells with 5×10⁵ viral genome equivalents of the GII.4-Sydney HuNoV-positive stool for 2 h, washing the cells extensively to remove unbound virus, subjecting 3 dpi cultures to two freeze-thaw cycles, pelleting and discarding cellular debris, and aliquoting supernatant. 5×10⁵ genome equivalents of this P0 stock were then applied to naïve B-cells and infections analyzed as described above.

HuNoV persistence studies. BJAB cells were inoculated with 5×10⁵ viral genome equivalents of the GII.4-Sydney HuNoV-positive stool. At 3 dpi, cells were washed three times to remove extracellular virus and re-plated at 5×10⁵ cells/mL. This procedure was repeated every 3 d. At each passage, one well was harvested for viral genome copy number determination.

HuNoV:bacteria incubation studies. Enterobacter cloacae was purchased from ATCC (ATCC 13047). Escherichia coli DH5-alpha from our common laboratory stock was used. Bacteria were inoculated into nutrient broth (NB) and incubated overnight at 37° C. Overnight cultures were serially diluted in NB to achieve the indicated concentrations and incubated at 65° C. for 40 min. for the purpose of heat inactivation. Inactivation was confirmed by plating on nutrient agar and inoculating into NB. For HuNoV studies, heat-inactivated bacteria were pre-incubated with virus for 1 h at 37° C. prior to inoculation onto cells. Lysates of heat-killed E. cloacae and E. coli were probed for H-type HBGA expression by western blotting with anti-H IgM antibody (Santa Cruz). Synthetic blood type H HBGA (GlycoTech product number 08-019; expected size of 238 kDa) was run in parallel as a positive control. The H-type antigen expressed by E. cloacae is ca. 63 kDa.

HuNoV attachment assay. A source of HuNoV is incubated with duplicate wells of BJAB-cells at 4° C. for the indicated amount of time. One sample is harvested without cell washing as an indication of input genome copy numbers; and one sample is centrifuged at 730 rcf (730 g) for 7.5 min. to pellet the cells with attached virus. The supernatant is removed and the pellet resuspended in cold PBS, and this wash is repeated two times. RNA is then extracted from both washed and unwashed sample, quantitative RT-PCR performed to determine viral genome copy numbers, and the amount of attachment determined by dividing genomes in washed sample by genomes in unwashed sample.

HuNoV antibody neutralization assays. A source of HuNoV is pre-incubated with a virus-specific antibody at 37° C. for 1 h and the sample is then tested in either attachment or infection assays, as described above. Serial dilutions of the antibody should be tested.

### Example 2

### Results for Murine Norovirus Infection of B-cells Studies

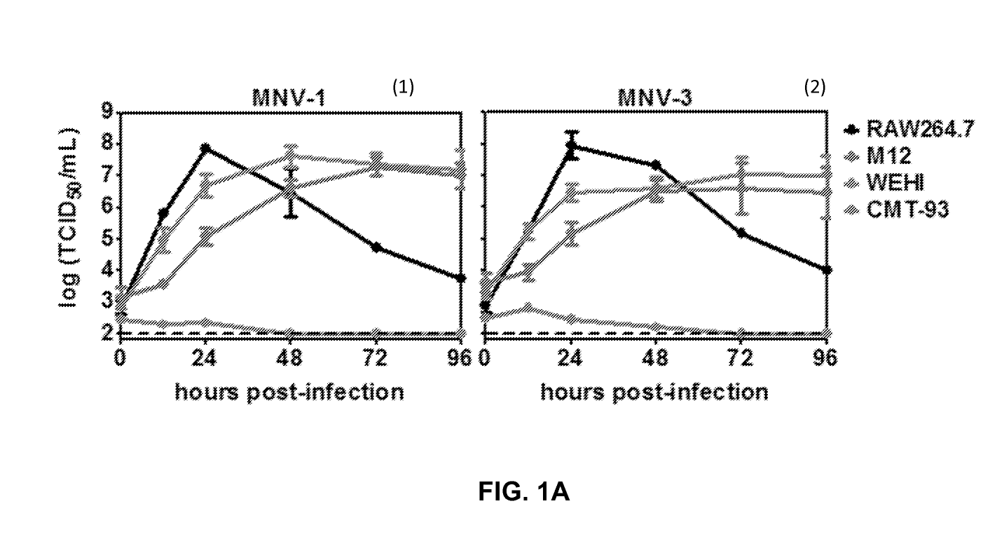

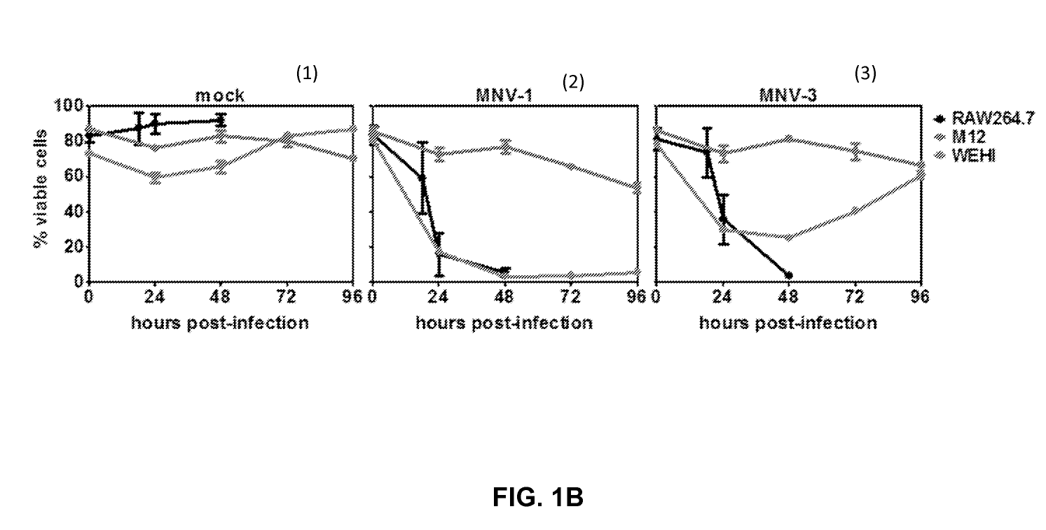

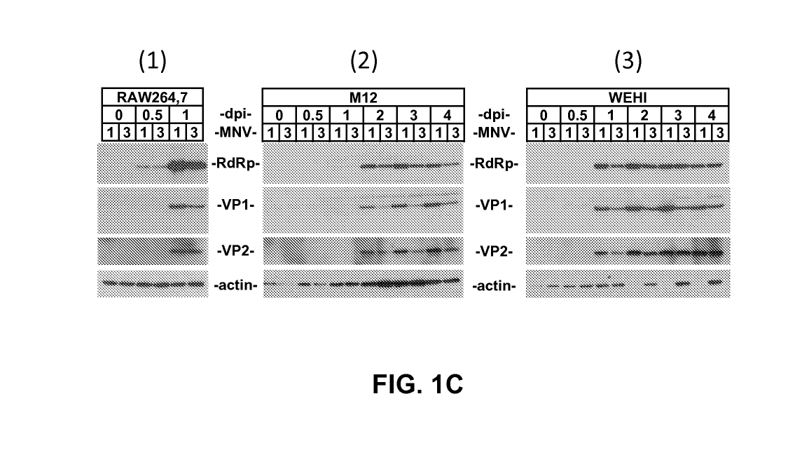

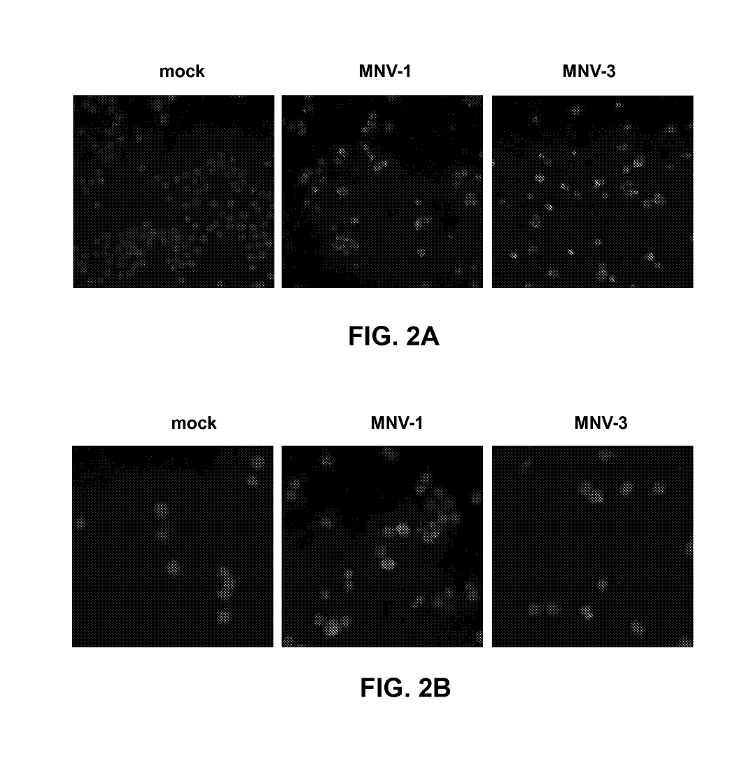

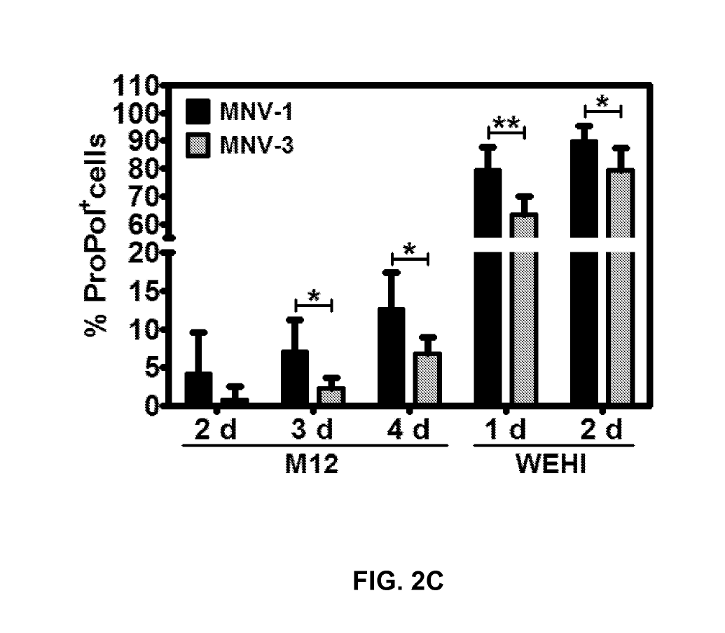

To test whether MNVs infect B-cells in culture, two murine B-cell lines called M12 and WEHI cells were infected with either MNV-1 or MNV-3 at MOI 5. The RAW 264.7 murine Mφ cell line known to be highly permissive to MNV infection was tested as a positive control (14). An IEC line CMT-93 was tested as a negative control since it has been shown that MNVs fail to infect IECs in vitro (9). As expected, RAW 264.7 cells produced ca. 10⁸ TCID₅₀ units per mL of each virus by 24 hpi whereas CMT-93 cells produced no detectable progeny virus through 96 h (FIG. 1A, 1 and 2). MNV-1 and MNV-3 both replicated efficiently in M12 and WEHI B-cells, although peak titers were not reached until 48-72 hpi (FIGS. 1A, 1 and 2). The M12 cells produced ca. 4-fold and 25-fold less peak MNV-1 and MNV-3 than RAW 264.7 cells, respectively; the WEHI cells produced ca. 2-fold and 8-fold less MNV-1 and MNV-3 than RAW 264.7 cells, respectively. Peak viral titers were slightly higher and occurred slightly faster in WEHI cells than M12 cells. While infection of RAW 264.7 and WEHI cells resulted in definitive cytopathic effect (CPE) upon microscopic analysis, no CPE was observed in M12 cultures (see below). Specifically, FIGS. 1A, 1 and 2 shows that murine B-cell lines support MNV infection. RAW 264.7 (black line), CMT-93 (green line), M12 (blue line), and WEHI (red line) cell lines were infected with either MNV-1 (1, left panel) or MNV-3 (2, right panel) at MOI 5. After 1 hr of infection on ice, cells were washed to remove unbound virus and incubated at 37° C. At the indicated times on the x-axis, supernatants were removed and titered on RAW 264.7 cells using a standard TCID₅₀ assay. Duplicate wells were tested for each cell line and each virus. The entire experiment was repeated three times. Data from all experiments were averaged and are reported as the log (TCID₅₀ units/mL). The limit of detection is indicated by a dashed line.

The extent of cell death induced by each virus in the two B-cell lines was next compared to RAW 264.7 cells. As expected based on the lytic nature of MNV infection in RAW 264.7 cells (14), there was nearly 100% loss of RAW 264.7 cell viability in cultures infected with either virus at 48 hpi (FIG. 1B; black lines). In stark contrast, MNV-infected M12 cells displayed only minimal cell death compared to mock-inoculated control cells (FIG. 1B; blue lines). WEHI cells displayed a more striking virus strain-specific phenotype, with MNV-1 causing nearly 100% cell death by 48 hpi but MNV-3 killing only 65% of the cells by this same time point; in the MNV-3-infected WEHI cultures, the surviving cells actually began to divide at 48-96 hpi (FIG. 1B; red lines). The same pattern of cell viability was observed in all three cell lines using trypan blue exclusion. Overall, M12 cells developed minimal to no CPE upon MNV infection whereas WEHI cells displayed a greater sensitivity to MNV-induced cell death that was virus strain-specific. Specifically, FIG. 1B shows that B-cell lines are differentially susceptible to MNV-induced CPE. RAW 264.7 (black lines), M12 (blue lines), and WEHI (red lines) cells were inoculated with mock inoculum (left panel, 1) or infected with MNV-1 (middle panel, 2) or MNV-3 (right panel, 3) at MOI 5. At 0, 24, 48, 72, and 96 hpi, duplicate wells per condition were stained with propidium iodide and the percentage of positive cells was determined. RAW 264.7 cells were only analyzed through 48 h of infection since no viable cells remained at this time point. The data are reported as the percentage of cells failing to incorporate dye over total cells (% viable cells) relative to the percentage of viable cells at 0 hpi. The entire experiment was repeated twice and values for all four replicates per condition averaged.

To further investigate the efficiency of MNV replication in B-cells, the kinetics of viral protein production was assessed using western blotting. The viral RNA-dependent RNA polymerase (RdRp), the major capsid protein VP1, and the minor structural protein VP2 were first detected in WEHI cells at 24 hpi (FIG. 1C, right panel, 3). In contrast, they were not detected in M12 cells until 48 hpi (FIG. 1C, middle panel, 2). Consistent with viral growth kinetics presented in FIG. 1A, the overall kinetics of viral translation was slower in B-cells than in RAW 264.7 cells where viral proteins can be detected by 12 hpi (FIG. 1C, left panel, 1). Two other observations were gleaned from these experiments: first, in all cell lines MNV-1 produced higher levels of RdRp, VP1, and VP2 than MNV-3. Second, there was no detectable actin in MNV-1-infected WEHI cells at 48-96 hpi although significant actin was detected in MNV-3-infected WEHI cells and M12 cells infected with either virus; these data are consistent with cell viability data presented in Fig. lB. Specifically, FIG. 1C shows that WEHI cells support faster MNV translation than M12 cells, and MNV-1 is translated more efficiently than MNV-3 in B-cells. RAW 264,7 WEHI and M12 cells were infected with either MNV-1 or MNV-3 at MOI 5. After 1 h on ice, cells were washed and incubated at 37° C. At the indicated hours post-infection (hpi), lysates from cells infected with the indicated virus were generated for the purpose of western blotting. Membranes were sequentially probed with anti-ProPol which recognizes the RdRp, anti-VP1, anti-VP2 and anti-actin. Duplicate wells were tested for each cell line and each virus. The entire experiment was repeated three times. One representative sample set is presented.

The infectivity of each MNV strain in RAW 264.7, M12, and WEHI cells was determined using IFA with a polyclonal antibody directed to the nonstructural protease-RdRp proteins (ProPol). As expected, nearly all RAW 264.7 cells infected with MNV-1 or MNV-3 at MOI 5 contained viral antigen at 24 hpi (FIG. 2A). However, only a small fraction of M12 cells infected with either virus strain at MOI 20 contained viral antigen at 96 hpi (FIG. 2B). There were a significantly higher number of antigen-positive cells in MNV-1-infected M12 cultures than in MNV-3-infected M12 cultures (FIG. 2C). In WEHI cultures infected at MOI 5, a majority of cells contained viral antigen at 48 hpi, similar to RAW 264.7 cells (FIG. 2C). As observed in M12 cells, MNV-1 infectivity was significantly higher than MNV-3 infectivity (FIG. 2C). Viral infectivity in RAW 264.7 and WEHI cells at later time points was not assessed due to the high degree of CPE in the cultures. The dramatic difference in M12 and WEHI infectivity correlated with viability results (see FIG. 1B); yet it was still surprising based on the fact that overall virus production in the two cell lines in single-step growth curves was similar (see FIG. 1A). Progeny virus produced in M12 cells was infectious - M12 supernatant-associated virus can be titered on RAW 264.7 cells; furthermore, high levels of virus were produced in a multi-step growth curve in which M12 cells were infected at MOI 0.05 (data not shown). Specifically, FIG. 2 shows that the relative MNV infectivity of B-cells is lower than in macrophages. (FIG. 2A) RAW 264.7 cells were inoculated with mock inoculum or infected with MNV-1 or MNV-3 at MOI 5. At 24 hpi, cells were stained with anti-ProPol and DAPI and imaged on a fluorescent microscope. The ProPol antibody was visualized with an Alexa Fluor 594-conjugated anti-goat secondary antibody. (FIG. 2B) The same experiment was performed for M12 cells except that cells were infected with MNV-1 or MNV-3 at MOI 20 and cells were stained at 48, 72, and 96 hpi (the representative images were generated at 96 hpi). (FIG. 2C) The percentage of virally infected cells in each B-cell line (M12 and WEHI) was quantified as the average ratio of ProPoi^{|} cells per total cells.

Specifically, viral antigen-positive cells and total cells based on DAPI stain were both counted in three random 40X fields per condition at the indicated time points. The entire experiment was repeated twice and all data per condition averaged.

### Example 3

### Murine Noroviruses Establish Persistent B-cell Infection

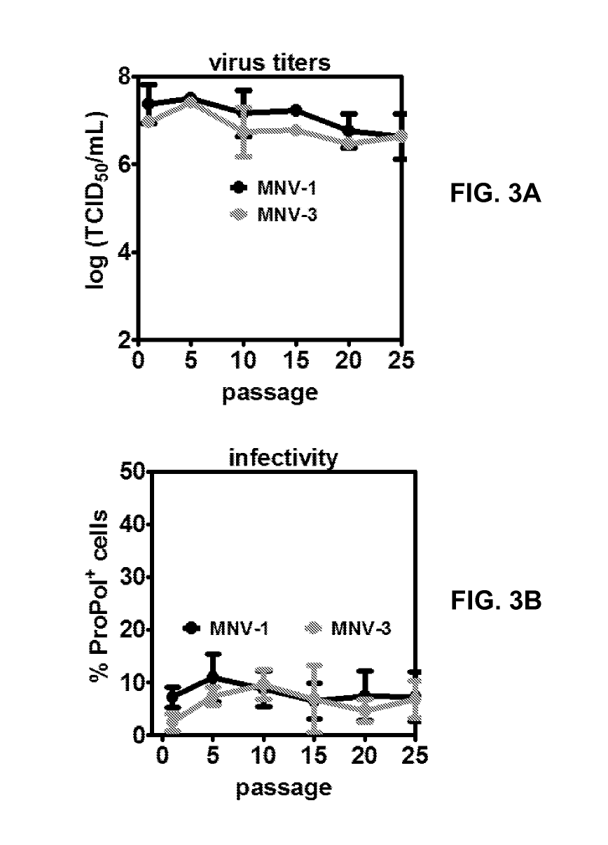

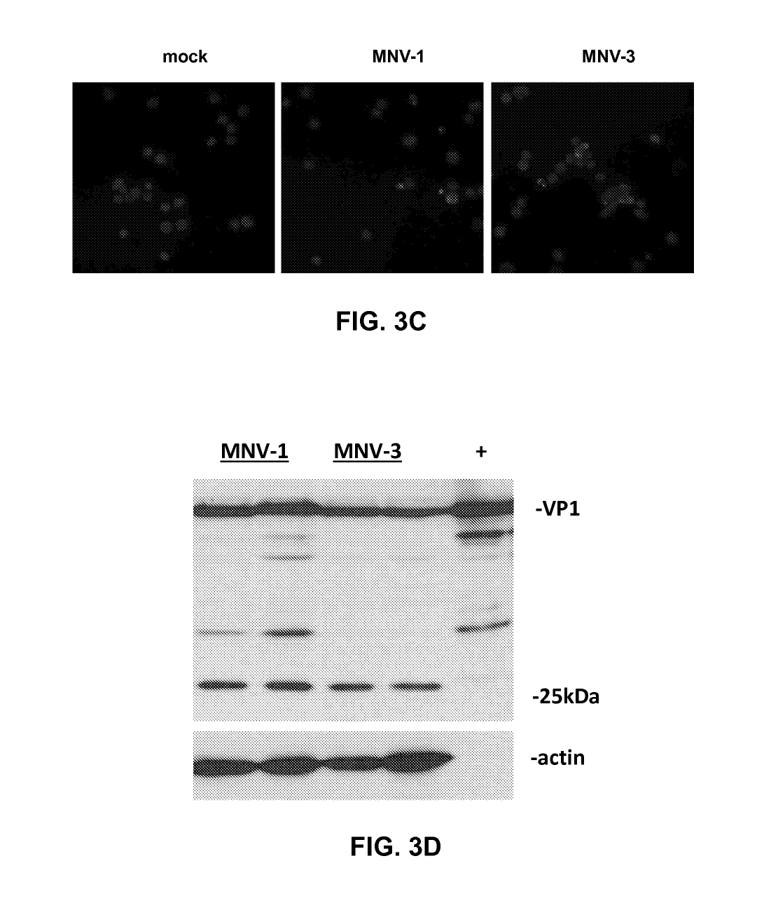

Because MNV infection of M12 cells was associated with minimal CPE, it was examined whether the cultures were capable of clearing infection or instead became persistently infected. To address this, M12 cells were infected with MNV-1 or MNV-3 at MOI 5 for 48 h. Cells were then washed extensively to remove extracellular virus and re-plated at a low cell density. This was repeated every 48 h for a total of 25 passages. In all passages tested, a comparable amount of supernatant-associated virus ranging from 2×10⁶-3×10⁷ TCID₅₀ units per mL for MNV-1 and 3×10⁶-2×10⁷ TCID₅₀ units per mL for MNV-3 was detected, demonstrating persistent M12 infection by both virus strains (FIG. 3A). MNV-3-infected WEHI cells were tested in parallel in one experiment and similar persistent infection through ten passages was observed. Viral infectivity during persistent M12 infection was also measured. Persistently MNV-1-infected cultures contained 7-11% viral antigen-positive cells over time, and persistently MNV-3-infected cultures contained 2-10% viral antigen-positive cells over time (FIG. 3B). Representative IFA images from passage 10 cultures are shown (FIG. 3C). At multiple passages, cell lysates were also probed for VP1 protein (FIG. 3D). The mature form of VP1 was present at significant quantity during persistent infection. Interestingly, there was also a unique 25 kDa form produced during persistent MNV-1 and MNV-3 infections of M12 cells that was not detected in virus stocks used for the initial inoculations (+; FIG. 3D); and which has not been observed in other cell lines (data not shown). It is interesting to speculate that this unique form of VP1 plays a role in persistence establishment and/or noncytopathic egress from M12 cells. Specifically, FIG. 3 shows that B-cells become persistently infected with MNV in vitro. Duplicate wells of M12 cells were infected with MNV-1 or MNV-3 at MOI 5 and passaged every 48 h. At the first passage and every fifth passage, the virus titers in the supernatants were determined using standard TCID₅₀ assay (FIG. 3A), and infectivity was analyzed by IFA using anti-ProPol antibody and DAPI (FIG. 3B); the percent infectivity was calculated as described in the description of FIG. 2. The entire experiment was repeated three times and all data per condition averaged. Representative images merging the viral ProPol signal (red) and the DAPI staining of nuclei (blue) are shown from passage 10 (P10) cultures (FIG. 3C). A representative western blot of cell lysates from persistently MNV-1- or MNV-3-infected M12 cultures (two independent cultures per virus strain) generated at passage 23 (P23) is shown (FIG. 3D). The MNV-1 virus stock used for initial infections was also tested (labeled as [+]). The blot was probed with anti-VP1 and re-probed for actin as a loading control.

### Example 4

### Peyer's Patch B-cells are Infected by MNVs in vivo

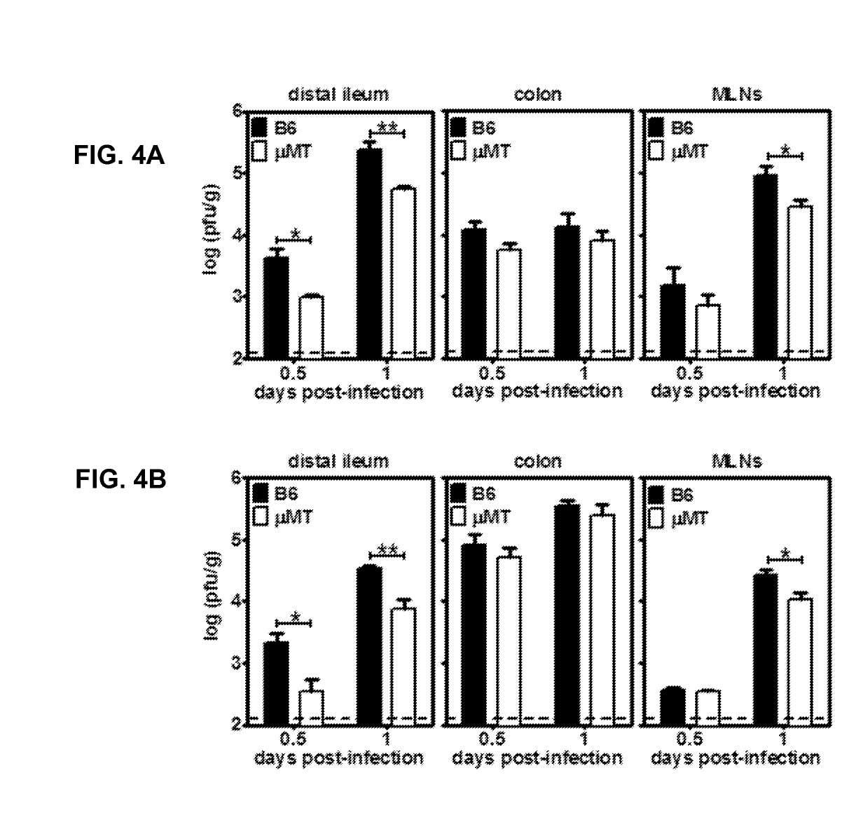

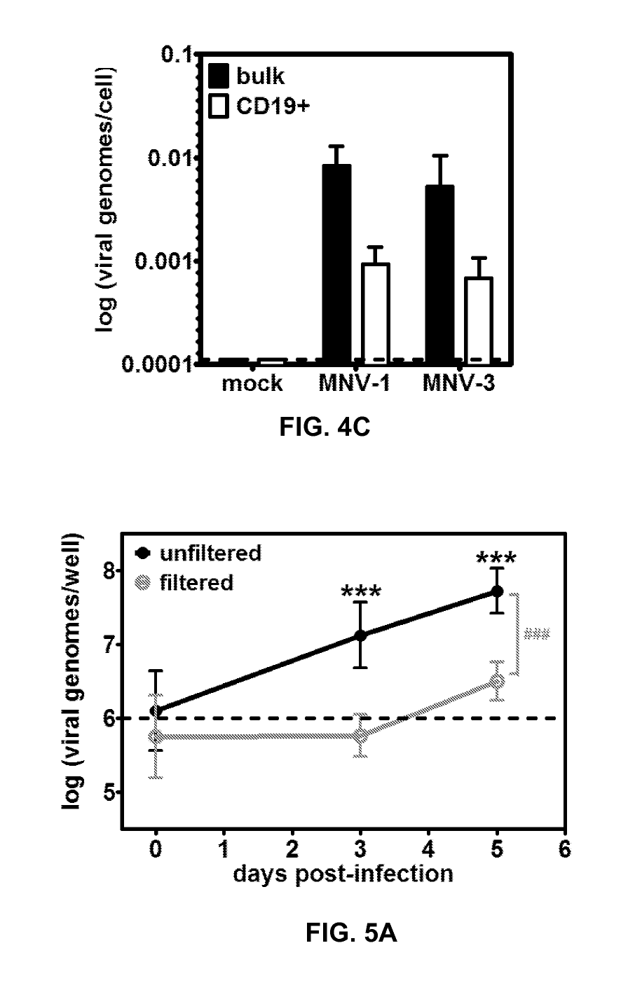

It has previously been reported that MNV-3 distal ileum titers are reduced in B-cell^{-/-} mice compared to wild-type mice at 24 hpi (19). To gain further insight into the significance of putative in vivo B-cell infection, titers of MNV-1 (FIG. 4A) and MNV-3 (FIG. 4B) in the distal ileum, colon, and mesenteric lymph nodes of wild-type (black bars) and B-cell^{-/-} (white bars) mice at 12 and 24 hpi were analyzed. Indeed, distal ileum titers of both virus strains were reduced at both time points in the absence of B-cells. In contrast, colon titers were comparable for both virus strains at both time points. In mesenteric lymph nodes, MNV-1 and MNV-3 titers were modestly reduced at 24, but not 12, hpi. Overall these data indicate that small, but not large, intestinal B-cells are targeted by MNV infection. Because Peyer's patches are localized along the small intestine, are known to contain high levels of infectious MNV-1 and MNV-3 (33), and are rich in B-cells, it was next examined whether Peyer's patch B-cells were infected. Specifically, CD19⁺ cells were purified from the Peyer's patches of mice infected with either MNV-1 or MNV-3. Viral genome was measured from bulk Peyer's patch cells or purified B-cells using qRT-PCR. Significant numbers of viral genomes can be detected in the bulk population and in the purified B-cells for both MNV-1 and MNV-3 (FIG. 4C). Specifically, FIG. 4 shows that Peyer's patch B-cells support MNV infection in vivo. Groups of C57BL/6 mice (black bars) and B-cell^{-/-} mice (white bars) were infected with 10⁷ TCID₅₀ units MNV-1 or MNV-3 and harvested at 0.5 and 1 dpi (n=10). Virus titers were determined by performing plaque assay on homogenates of the indicated tissues. The data are presented as plaque forming units (pfu) per gram tissue on a logarithmic scale and data for all mice in each group were averaged. Limits of detection are indicated by the dashed lines. The C57BL/6 and B-cell^{-/-} titers at each condition were compared for statistical analysis. (FIG. 4C) Groups of C57BL/6 mice (n=5-8) were inoculated with either mock inoculum or 10⁷ TCID₅₀ units MNV-1 or MNV-3 perorally. At 1 dpi, Peyer's patches were dissected from the small intestine and single cell suspensions generated. RNA was extracted from either bulk Peyer's patch cells (black bars) or magnetically separated CD19^{|} B-cells (white bars; purified to>97% purity). Quantitative RT-PCR was performed using virus-specific and GAPDH-specific primers. Data are reported as the log of [viral genomes/cell. The experiment was repeated three times and data from all experiments averaged. The limit of detection is indicated by a dashed line.

Discussion pertaining to MNV infection of B-cells: It was demonstrated that MNV-1 and MNV-3 infected both an immature (WEHI) and a mature (M12) B-cell line. Infection of immature B-cells was more efficient than infection of mature B-cells in terms of overall progeny virus production, kinetics of replication, and viral protein production (FIG. 1). Furthermore, the infectivity of immature B-cell infection was dramatically higher than in mature B-cells (FIG. 2). The magnitude of the difference in WEHI and M12 infectivity seems incongruous with the modest difference in overall virus production but it is important to recognize that peak viral titers are delayed ca. 24 h in M12 cells compared to WEHI cells.

The low infectivity of M12 infection correlated with an apparent absence of CPE (FIG. 1B), a remarkable observation considering that NoVs are nonenveloped and are known to lytically infect other cell types (14). A number of other nonenveloped viruses can nonlytically infect cells in culture (36-40). For example, in a recent paradigm-shifting report, noncytopathic nonenveloped hepatitis A virus (HAV) was shown to acquire a cellular envelope presumably as a mechanism to evade recognition by the host immune response; it was reported that HAV is released in enveloped form in vitro and in vivo (41). Hepatitis A virus is a member of the Picornaviridae family which shares many features of genomic organization and replication strategy with the Caliciviridae family of which NoVs segregate. The possibility that MNV infection of M12 cells is in fact cytopathic cannot currently be ruled out, but this is unapparent in the assays due to the low MNV infectivity in this cell type. Although differences in MNV infection of immature and mature B-cells was observed, it also cannot unequivocally be concluded that B-cell maturation state correlates with NoV cytopathic potential until multiple other B-cell lines of varying states are tested. However, in preliminary studies it was determined that infection of another mature B-cell line called A20 is noncytopathic. During natural infections, it seems likely that mature B-cells are more physiologically relevant targets since intestinal B-cells are expected to have gone through development in the bone marrow prior to trafficking to the intestine. However, MNVs reach the MLNs and some strains even replicate efficiently in the spleen where immature B-cells can be found. In fact, it has recently been demonstrated that a small number of immature B-cells localize to the intestinal lamina propria (42), suggesting that immature B-cells may be targeted during NoV infections as well.

The distinct nature of M12 cell infection correlated with the ability of MNVs to establish persistence in this cell line. This persistent infection was associated with ongoing progeny virion production since 10⁶-10⁷ TCID₅₀ units per mL of supernatant-associated virus were detected throughout 25 passage events (FIG. 3A). The infectivity data revealed that only a fraction of cells in infected M12 cultures supported ongoing MNV replication at any given time since 2-11% of cells stained positive for viral nonstructural protein throughout persistent infection (FIG. 3B). These results are strikingly similar to recent work on persistent coxsackievirus B3 (CVB3) infection of astrocytic cells where low infectivity correlated with minimal expression of the canonical CVB3 receptors (43). One possible contributor to persistent M12 infection by MNVs is cell type-specific differences in receptor expression, although MNV entry receptors have yet to be defined. MNV-3 infection of WEHI cells was initially cytopathic and caused cell death in ca. 65% of the culture (FIG. 1B); however, the cells surviving initial infection failed to completely clear virus and instead became persistently infected similar to M12 cells. On the other hand, MNV-1 cultures were completely susceptible to initial lytic infection (FIG. 1B). This virus strain-specific distinction may be explained by the fact that MNV-1 more efficiently antagonizes host cytokine production via the activity of its virulence factor 1 (VF1) protein than MNV-3 (19, 44). Persistent infection correlated with the detection of a small ca. 25 kDa form of the VP1 capsid protein in infected cells. This form of VP1 was not detected in virus stocks used for initial infections (FIG. 3D; [+]) or in the supernatants of infected M12 cultures so it likely does not become incorporated into progeny virions. It is possible that VP1 regulates a noncytopathic form of viral egress. Alternatively, VP1 may interfere with productive virus assembly, thereby reducing replication efficiency to a level where persistent infection can be established in a culture. Finally, VP1 may be anti-apoptotic and thus increase cell survival upon MNV infection.

Finally, it is confirmed that B-cells are MNV targets during enteric infections. There appears to be intestinal specificity to the cell tropism of MNVs with B-cells being targeted in the small intestine (e.g., distal ileum) but not the large intestine (e.g., colon). Indeed, Peyer's patch B-cells contained significant numbers of viral genomes at the peak of acute infections, correlating with decreased viral titers in RAG1^{-/-} and B-cell^{-/-} mice compared to wild-type mice at this time point (FIGS. 4A and 4B).

Overall it is concluded that MNVs efficiently infect multiple B-cell lines in vitro and Peyer's patch B-cells in vivo. The nature of infection is distinct from infection of other known cellular targets of MNVs such as Mφs and DCs. Of particular importance, mature B-cells display minimal CPE; support a low frequency of MNV infectivity; and are persistently infected in vitro which can have significant implications on NoV pathogenesis in vivo.

### Example 5

### Human Norovirus Direct Infection of B-Cells

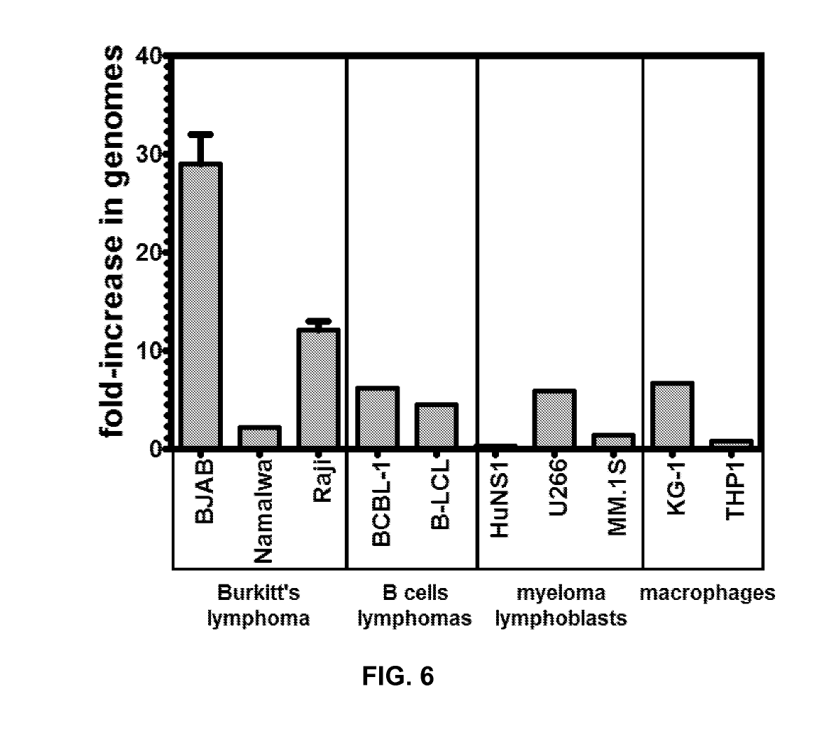

10⁶ genome equivalents of a stool sample collected from a GII.4 HuNoV-infected human patient were added directly to human B-cells, either unfiltered or filtered through a 0.2 micron membrane. At 0, 3, and 5 days post-infection (dpi), cells and supernatants were collected, the cells were frozen/thawed to release intracellular virus, cell debris was spun out, and RNA was extracted from lysate. Quantitative RT-PCR was then performed using primers specific to the virus. In FIG. 5A, the total viral genome copy numbers per well are indicated. The fact that unfiltered stool, but not filtered stool, supported viral replication indicated that enteric bacteria may facilitate the infection (the 0.2 micron filters used eliminate bacteria but not smaller viruses)—this can be direct (e.g., the virus binds the bacteria and this enhances receptor engagement; this type of mechanism has been shown for poliovirus recently) or indirect (e.g., a bacterial substance, such as LPS, could activate the B-cells and the virus replicates more efficiently in activated cells). The fact that UV treatment of unfiltered stool (UV-GII.4) prevented viral replication is an important control since UV treatment should inactivate the virus and strongly inhibit genome replication (FIG. 5B). In FIG. 5C, cell lysates of cultures inoculated with PBS (mock) or unfiltered GII.4-positive stool, at 0, 3 and 5 dpi were used in western blotting probing with an antibody specific to the HuNoV nonstructural 6 (NS6) protein. Cells at 0 and 5 dpi were also stained with anti-capsid (VP1) antibody and analyzed by IFA (FIG. 5D). We were able to detect both NS6 and capsid proteins in the GII.4-infected BJAB cells using these techniques, demonstrating the virus infects B-cells, is translated to produce nonstructural proteins, replicates its genomes, and synthesizes structural protein. Confirming that infectious virus is produced in BJAB cells, supernatants transferred from the initial infected culture to naïve BJAB cells initiated a second round of replication (data not shown). However, BJAB cells did not become persistently infected like MNV-infected M12 cells (data not shown). To test whether the BJAB cell line is unique in its ability to support HuNoV infection or whether this is a common feature of human B-cell lines, we infected a panel of human B-cell and macrophage cell lines with the unfiltered GII.4-positive stool and measured viral genome replication between 0 and 3 dpi. BJAB cells supported maximal viral infection, Raji cells supported lower levels of reproducible infection, and all other cell lines failed to support significant viral replication (see FIG. 6).

### Example 6

### Human Norovirus Co-Culture Infection of B-Cells

In this experiment, human intestinal epithelial cells (IECs; HT-29 cell line) were cultured on the membrane of a transwell, confirmed to be confluent using a FITC-dextran diffusion assay, and human BJAB cells were added to the lower chamber (as shown in FIG. 7A). The GII.4-positive stool was added to the apical supernatant of these cultures, and samples were collected at 0 and 3 dpi and assessed for levels of viral genomes. While no viral genome increase was detected in the apical supernatants or IEC compartments (data not shown), a significant increase was detected for in the underlying B-cell compartment when unfiltered, but not filtered, stool was used as inoculum (FIG. 7B). Importantly, detection of viral genomes in the basal chamber does not simply reflect passive diffusion of input virus across the confluent epithelial barrier because parallel infections with unfiltered GII.4-positive stool in the absence of BJAB cells in the basal chamber were negative for viral genome replication. These data demonstrate that the virus can be transported across an epithelium to access B-cells—relevant to how the virus may access B-cells from the intestinal lumen in vivo.

### Example 7

### HBGA and Bacterial Stimulation of Human Norovirus Infection of B-Cells

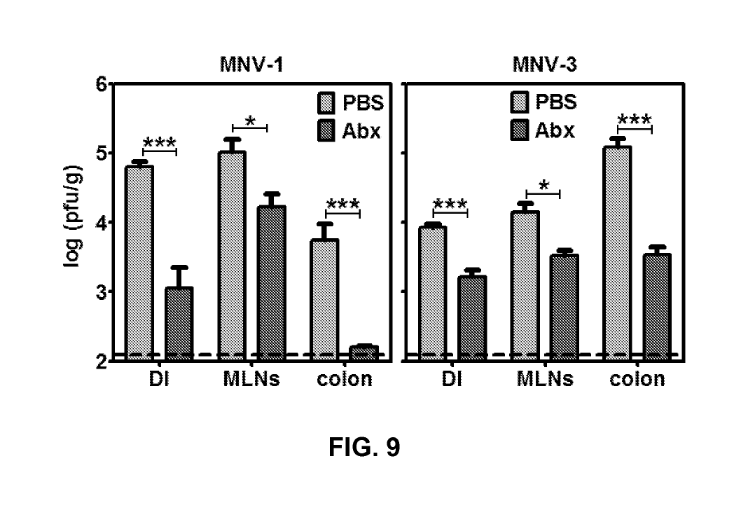

Based on our observations that filtration of a HuNoV-positive stool sample ablates viral infectivity (see FIG. 5A and 7B), we next probed the hypothesis that commensal bacteria stimulate HuNoV infection of B-cells. To this end, we tested whether infectivity of filtered stool could be rescued by pre-incubation with a specific bacteria. HuNoVs are known to bind HBGAs on human cells. Certain bacteria express HBGA-like carbohydrates on their surface while immune cells do not express these glycans. Thus, we reasoned that commensal bacteria might provide HuNoVs with stimulatory glycans required to infect B-cells. The GII.4-Sydney HuNoV strain is known to bind a variety of HBGAs, including A, B, and H antigens, which are expressed by the commensal bacteria Enterobacter cloacae. When filtered GII.4 HuNoV-positive stool was incubated with heat-killed E. cloacae prior to inoculation of BJAB cells in either direct infection or co-cultures, infectivity was completely restored (FIG. 8A and 8B). These data support the idea that HBGA-like carbohydrates, such as bacteria expressing the correct HBGA for the HuNoV strain in question, facilitate HuNoV infection of B-cells. Further support that commensal bacteria enhance or facilitate NoV infections was shown in in vivo studies of MNV. When mice were depleted of a majority of their commensal gut flora through oral antibiotic treatment, MNV infections were significantly reduced as indicated by significant reductions in viral titers in multiple tissues (FIG. 9).

### Example 8

### Human Norovirus B-Cell Attachment Assay and Antibody Neutralization Assays

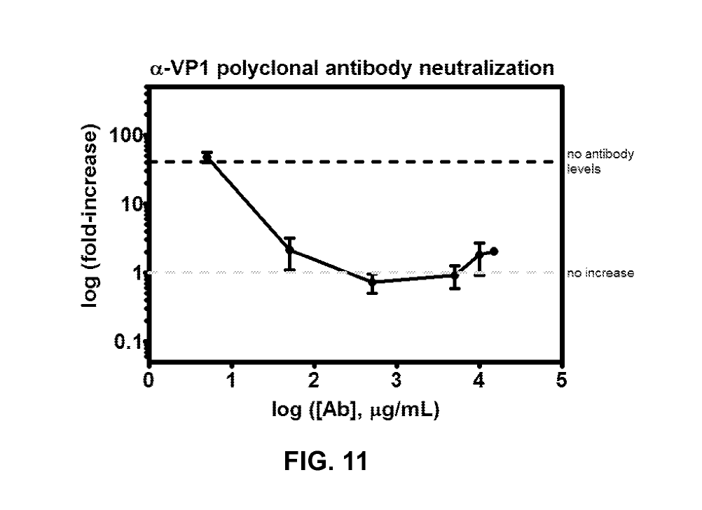

To probe the mechanism by which HBGA facilitate HuNoV infection of B-cells, we next performed attachment assays. As was observed for B-cell infections, filtration of the GII.4-positive stool ablated viral attachment to the surface of BJAB cells while pre-incubation of filtered stool with synthetic H completely restored attachment (FIG. 10A). Further evidence that the H-type HBGA specifically stimulates viral attachment, pig gastric mucin (PGM) was able to restore attachment of virus in filtered stool (FIG. 10B) and treatment of unfiltered stool with a-fucosidase (an enzyme that specifically removes the fucose moiety of H-type antigen that is bound by GII.4 HuNoVs) significantly reduces viral attachment (data not shown). We have also determined that pre-incubation of filtered stool with a polyclonal anti-VP1 antibody ablates viral attachment to B-cells (data not shown). Similarly, pre-incubation of unfiltered stool with this antibody completely prevents viral infection of B-cells (FIG. 11), validating the utility of this infection system to study functionality of anti-HuNoV antibodies. This will be an invaluable tool in validating correlates of protective immunity in vaccine cohorts, for example.

Discussion pertaining to HuNoV infection of B-cells and commensal bacteria as a co-factor for NoV infections. Our identification of B-cells as a target for HuNoV infection represents the first identification of a permissive cell type in vitro for these critical human pathogens. When a GII.4-Sydney HuNoV-positive stool sample was applied to the human B-cell line BJAB, we detected significant albeit modest increases in viral genome copy number. As expected, viral genome replication was completely ablated by UV treatment and a polyclonal α-VP1 antibody fully neutralized infectivity. Further confirming viral replication in these cells, we detected nonstructural and structural protein. Most importantly, lysates from the infected cells supported infection of naïve BJAB cells, demonstrating productive infection of progeny virions. Similar to MNV infection of M12 cells, we failed to detect CPE in HuNoV-infected BJAB cultures microscopically or using trypan blue exclusion (data not shown). Although both MNV and HuNoV infection of certain B-cell lines is thus apparently noncytopathic, GII.4-Sydney HuNoV did not establish persistence in BJAB cells as MNVs did in M12 cells.

Filtration of the GII.4-Sydney HuNoV-positive stool sample through a 0.2-μm membrane significantly reduced viral genome replication in direct B-cell infection and in the transwell system, revealing a filterable co-factor for HuNoV infection of B-cells. Because we used unprocessed stool as a source of virus, we reasoned that the most likely candidate for a filterable co-factor was commensal bacteria. H antigen-expressing E. cloacae stimulated viral infection of B-cells in a dose-dependent manner whereas H antigen-negative Escherichia coli and free lipopolysaccharide (LPS) failed to stimulate infection. The bacteria used in these experiments were heat-killed prior to incubation with the filtered stool inoculum so bacterial viability is not required for its stimulatory activity. Providing further evidence that the HBGA specifically stimulates infection, synthetic H antigen also rescued infectivity of filtered virus inoculum. Although we do not yet know the precise mechanism by which a HBGA (either free or bacterial-bound) stimulates HuNoV infection of B-cells, we determined that it facilitates viral binding to the surface of B-cells.

Antibiotic depletion of the intestinal microbiota prior to MNV-1 and MNV-3 infections resulted in significant reductions in acute virus titers. These data strongly argue that NoVs have evolved common strategies to use bacterial ligands to enhance their infectivity although the strategies used by MNVs and HuNoVs are likely not identical. For example, MNVs can efficiently infect macrophages, dendritic cells, and B-cells in vitro in the absence of bacteria. Moreover, mice do not express HBGAs so MNVs must use distinct bacterial ligands. Regardless, the mouse model will be invaluable in dissecting the general in vivo relevance of these interactions to overall NoV infection. Based on our collective data, we have generated a working model of NoV infection whereby virus binds commensal bacteria in the gut lumen via a bacterial carbohydrate, the virus-bacteria or virus-glycan complex is transcytosed across the intestinal epithelium, and the bacterial glycan stimulates viral infection of underlying immune cells (65).

The availability of a HuNoV cultivation system will enable the development of tools/systems for investigating mechanisms of viral replication methods for measuring immune correlates of protection (e.g., antibody neutralization assays), development of live attenuated viral vaccine candidates, and testing of putative antiviral compounds in vitro. In fact, our finding that a polyclonal α-VP1 antibody could fully neutralize viral infectivity provides proof-of-principle that this new cell culture system can be used to assess true neutralizing activity of α-HuNoV antibodies.

### Example 9

### Detailed Human Norovirus Co-Culture System. (Transwell in vitro cell Culture System)

Media
DMEM (complete)
DMEM (Fisher Scientific)
10% FBS
1× Pen/strep
RPMI (10%)
RPMI (Fisher Scientific cat# MT10040CM)
10% FBS
1× Pen/strep
None of the RPMI media for BJABs contain BME.
BJAB culturing:

From Frozen Stock: Vial removed from LN2 freezer was thawed in a 37° C. water bath until cells were just thawed. Cells were added to 9 ml of RPMI and centrifuged at 1100 rpm for 10 min. Medium was aspirated and pellet was re-suspended in 1 ml of RPMI with 50% FBS. A small aliquot (10 μl) was taken and used to determine cell concentration. Culture was then seeded into wells of a 6 well plate at a density between 5-7×10⁵ cells per well. Additional RPMI with 50% FBS was added to the wells to bring the final volume to 2 ml. Plate was incubated at 37° C.

1^{st} Passage: The culture was checked 3 days after initial plating. A growing, healthy culture formed clumps of cells. Initially, smaller clumps (10-20 cells) were seen, but after 1-2 days of additional incubation at 37° C., larger clumps (40+) cells began to form. When the culture concentration reached 1-2×10⁶ cells/ml, it was split. Seed 5×10⁵ cells per well of a 6 well plate. Added additional RPMI with 1% FBS to bring final volume to 2 ml. Plate was incubated at 37° C.

Subsequent Passaging: On average, a culture reached 1×10⁶ cells per well after 3-4 days of culture. When seeded at 5×10⁵ cells per well, cells can be incubated for up to 5 days. Cells can be passaged after 3-4 days of incubation.
HT-29 culturing:

From Frozen Stock: Vials of cells were thawed in a 37° C. water bath until cells were just thawed. Cells were added to 9 ml of DMEM and centrifuged at 1100 rpm for 10 min. Medium was aspirated and pellet was resuspended in 1 ml DMEM. The cells were seeded into a T-25 flask with 7 mL DMEM. The flask was incubated at 37° C. Media was changed every 4-5 days until culture was 80-90% confluent. 1^{st} Passage: When culture was 80-90% confluent, the cell layer was rinsed with 1X PBS to remove all traces of serum which contains trypsin inhibitor. 2 mL of trypsin was added and cells were observed under an inverted microscope until the cell layer was dispersed (usually within 5 to 15 minutes). Cells that are difficult to detach may be placed at 37° C. to facilitate dispersal.

6.0 to 8.0 mL of complete growth medium was added and cells aspirated by gently pipetting. Cells were counted and appropriate aliquots of the cell suspension were added to new culture vessels. At this point the culture was expanded to T-75 flasks. The recommended inoculum is 1×10⁴ viable cells/cm². Cultures were incubated at 37° C., 5% CO₂.

Subsequent Passaging: When the flask reached 80-90% confluency, cells were rinsed and treated with trypsin as above. Cells were split at 1:4 to 1:8. Cultures were incubated at 37° C., 5% CO₂.

Seeding to Hanging Wells: When a flask of cells was 80-90% confluency, the cells were rinsed with PBS, treated with trypsin and resuspended in DMEM as above. 1 ml of DMEM was added to the wells of a 12 well plate. Aseptically, hanging wells were transferred to the 12 well plate and 200 μl of DMEM. 5×10⁴ cells per hanging well were seeded and final volume was brought to lml with DMEM. The plate was incubated at 37° C., 5% CO₂. Media above and below the hanging well was changed every 3-4 days. Once the cells in the well were 100% confluent for one week, they were tested for polarization of cells using the dextran-FITC assay.

Virus Prep

Frozen stool samples obtained from CDC were incubated in a 37° C. water bath until just thawed and then distributed into 50 μl aliquots and stored at −80° C. One aliquot was removed and used to titer the virus stocks.

Experimental Design

BJABs: Cells from multiple culture wells were combined into a 50 ml conical tube. The tube was gently mixed and the cell concentration determined. Wells of a 12-well plate were then seeded with 2.5×10⁵ cells. The culture was then brought up to a final volume of 1300 μl using RMPI (10% FBS). Cultures used had been passed no less than 3 days prior to the start of the experiments.

HT-29s: Using aseptic technique, old media was removed from the hanging wells and transfered to the well of a 12 well plate seeded with BJABs. 200 μl of DMEM was added.

Virus: An aliquot (50 μl) of virus was removed from −80° C. storage and incubated in a water bath until just thawed. The virus was then placed on ice until use, then seeded onto the apical side of the hanging wells. Plates were incubated at 37° C.

At designated time points, cells were harvested and distributed into microcentrifuge tubes for further analysis. Supernatants were removed from the apical side of hanging wells, then the well was removed and the media and cells on the basolateral side were removed. Finally 1 ml of PBS was added to the hanging well and the HT-29 cells were scraped off and transferred. All aliquots were stored at −80° C.

Sample Analysis

RNA extraction: Sample aliquots (500 μl) were removed from −80° C. and thawed in a 37° C. water bath. Once thawed, samples were returned to −80° C. This process was repeated 1 more time (for a total of 3 freeze/thaw cycles).

After the final thaw, 1 ml of Trizol was added to the culture and incubated at room temperature for 5 min. Then 200 μl of chloroform was added and the sample was vortexed and incubated at room temperature for 3 min. Samples were then centrifuged at 12,000× g for 15 min. The aqueous phase was put into a fresh tube and 10 μg of glycerol was added to each sample. After mixing, 500 μl of isopropanol was added and then the samples were incubated at room temperature for 10 min at 4° C. at 12,000× g. The sample was centrifuged again for 10 min (at 4° C.). Isopropanol was removed and pellet was washed with 75% ethanol. Samples were centrifuged again for 5 min at 7,500× g. Ethanol was removed and pellet allowed to air dry for 5-10 min. Then pellet was re-suspended in 30 μl of pre-warmed (70° C.) RNase/DNase free water.

cDNA amplification: The Superscript kit (Invitrogen # 11752-050) was used per the manufacturer's instructions. 500 ng of RNA was added to the sample reactions.

qPCR: Sybr green (Fisher Scientific # FERK0243) was used according to the manufacturer's instructions. Primers NK2PF (5′-ATGTTYAGRTGGATGAGATTCTC) (SEQ ID NO: 3) and NK2PR (5′-TCGACGCCATCTTCATTCAC) (SEQ ID NO: 4) were used to amplify GII viruses at a final concentration of 200 nM each. Primers used for GI viruses were NK1PF (5′-GCYATGTTCCGYTGGATG) (SEQ ID NO: 5) and NK1PR (5′-GTCCTTAGACGCCATCATCAT) (SEQ ID NO: 6) at the concentrations mentioned above. 2 μl of undiluted cDNA was added to each sample well and each sample was run in triplicate. Samples were amplified on a Biorad iCycler under the following cycling conditions: 95° C. for 10 min followed by 41 cycles of 95° C. for 15s, 58° C. for 30 s, and 72° C. for 30 s. Melt curve was generated at 65° C. for 10 s for 61 cycles.

As an alternative, a one step RT-qPCR using the AgPath One-Step RT-PCR Kit (Ambion) can be used. In this alternative technique, a master mix can be set up and the reaction performed according to the manufacture's instructions (adding 150 ng of RNA per PCR well). The following primers and probes can be use with the following cycling program:

Forward Primer (NKP2F):
(SEQ ID NO: 3)
ATG TTY AGR TGG ATG AGA TTC TC
Reverse Primer (NKP2R):
(SEQ ID NO: 4)
TCG ACG CCA TCT TCA TTC AC
GII.4 Probe (RING2-TP):
(SEQ ID NO: 7)
FAM-TGG GAG GGC GAT CGC AAT CT-TAMARA

Cycling program:

10 min at 42° C. (RT step)

10 min at 95° C.

45 cycles of:
15 s at 95° C.
60 s at 60° C.

Collect fluorescence data during the “60 s at 60° C.” step.

### Example 10

### Detailed Human Norovirus Direct Infection System. (In vitro Cell Culture System)

Media

RPMI (50%)

RPMI (Fisher Scientific cat# MT10040CM)

50% FBS

1× Pen/strep

RPMI (10%)

RPMI (Fisher Scientific cat# MT10040CM)

10% FBS

1× Pen/strep

None of the RPMI media for BJABs contain BME.

BJAB culturing:

From Frozen Stock: Vials of cells were thawed in a 37° C. water bath until cells were just thawed. Cells were added to 9 ml of RPMI and centrifuged at 1100 rpm for 10 min. Medium was aspirated and pellet was re-suspended in lml of RPMI with 50% FBS. A small aliquot (10 μl) was taken and used to determine cell concentration. Culture was then seeded into wells of a 6 well plate at a density between 5-7×10⁵ cells per well. Additional RPMI with 50% FBS was added to the wells to bring the final volume to 2 ml and the plate was incubated at 37° C.

1^{st} Passage: The culture was checked 3 days after initial plating. A growing, healthy culture begins to form clumps of cells. Initially, smaller clumps (10-20 cells) were seen, but after 1-2 days of additional incubation at 37° C., larger clumps (40+) cells began to form. When the culture concentration reaches 1-2×10⁶ cells/ml, the culture was split. 5×10⁵ cells per well were seeded into a six well plate. Additional RPMI with 1% FBS was added to bring final volume to 2 ml and the plate incubated at 37° C. Subsequent Passaging: Subsequent Passaging: On average, a culture reached 1×10⁶ cells per well after 3-4 days of culture. When seeded at 5×10⁵ cells per well, cells can be incubated for up to 5 days. Cells can be passaged after 3-4 days of incubation.

Virus Prep

Stool samples obtained from CDC were incubated in a 37° C. water bath until just thawed and then distributed into 50 μl aliquots and stored at −80° C. One aliquot was removed and used to titer the virus stocks.

Experimental Design

BJABs: Cells from multiple culture wells were combined into a 50 ml conical tube. The tube was gently mixed and the cell concentration determined. Wells of a 12-well plate were then seeded with 2.5×10⁵ cells. The culture was then brought up to a final volume of 1300 μl using RMPI (10% FBS). Alternatively, wells of a 48-well plate can be seeded with 6.45×10⁴ cells per well and the culture volume brought up to 1000 μl using RMPI. Cultures used had been passaged no fewer than 3 days prior to the start of the experiments.

Virus: An aliquot (50 μl) of virus was removed from −80° C. storage and incubated in a water bath until just thawed. The virus was then placed on ice and 450 μl of RPMI (10% FBS) was added to the vial. Virus was seeded into wells at a concentration of 1×10⁵-1×10⁶ genome copies per well. Plates were incubated at 37° C. At 1 hr, 3 and 5 days post infection, cells from one well were harvested and distributed into microcentrifuge tubes in 2-500 μl and 1-300 μl aliquots. Aliquots were stored at −80° C.

Alternatively, cells can be infected in a 1.5 mL microcentrifuge tube seeded 6.45×10⁴ BJAB cells per reaction. In this infection technique, frozen stool is removed from −80° C. storage, thawed and diluted 1:10 in 10% RPMI. 1×10⁵-1×10⁶ genome copies of virus per reaction is added to the BJAB cells. The mixture is incubated for 2 hrs and then centrifuged at 730 rcf (730 g) for 7.5 min and the supernatent removed. The pellet is resuspended in 50-100 μl of RPMI per reaction and distributed among an appropriate number of wells. The total volume of each well is brought up to 1000 μl.

### Sample Analysis

RNA extraction: Sample aliquots (500 μl) were removed from −80° C. and thawed in a 37° C. water bath. Once thawed, samples were returned to −80° C. This process was repeated 1 more time (for a total of 3 freeze/thaw cycles).

After the final thaw, 1 ml of Trizol was added to the culture and incubated at room temperature for 5 min. Then 200 μl of chloroform was added and the sample was vortexed and incubated at room temperature for 3 min. Samples were then centrifuged at 12,000× g for 15 min. The aqueous phase was put into a fresh tube and 10μg of glycerol was added to each sample. After mixing, 500 μl of isopropanol was added and then the samples were incubated at room temperature for 10 min at 4° C. at 12,000× g. The sample was centrifuged again for 10 min (at 4° C.). Isopropanol was removed and pellet was washed with 75% ethanol. Samples were centrifuged again for 5 min at 7,500× g. Ethanol was removed and pellet allowed to air dry for 5-10 min. The pellet was then re-suspended in 30 μl of pre-warmed (70° C.) RNase/DNase free water.

cDNA amplification: The Superscript kit (Invitrogen # 11752-050) was used per the manufacturer's instructions. 500 ng of RNA was added to the sample reactions.

qPCR: Sybr green (Fisher Scientific # FERK0243) was used according to the manufacturer's instructions. Primers NK2PF (5′-ATGTTYAGRTGGATGAGATTCTC) (SEQ ID NO: 3) and NK2PR (5′-TCGACGCCATCTTCATTCAC) (SEQ ID NO: 4) were used to amplify GII viruses at a final concentration of 200 nM each. Primers used for GI viruses were NK1PF (5′-GCYATGTTCCGYTGGATG) (SEQ ID NO: 5) and NK1PR (5′-GTCCTTAGACGCCATCATCAT) (SEQ ID NO: 6) at the concentrations mentioned above. 2 μl of undiluted cDNA was added to each sample well and each sample was run in triplicate. Samples were amplified on a Biorad iCycler under the following cycling conditions: 95° C. for 10 min followed by 41 cycles of 95° C. for 15 s, 58° C. for 30 s, and 72° C. for 30 s. Melt curve was generated at 65° C. for 10 s for 61 cycles.

As an alternative, a one step RT-qPCR using the AgPath One-Step RT-PCR Kit (Ambion) can be used. In this alternative technique, a master mix can be set up and the reaction performed according to the manufacture's instructions (adding 150 ng of RNA per PCR well). The following primers and probes can be use with the following cycling program:

Forward Primer (NKP2F):
(SEQ ID NO: 3)
ATG TTY AGR TGG ATG AGA TTC TC
Reverse Primer (NKP2R):
(SEQ ID NO: 4)
TCG ACG CCA TCT TCA TTC AC
GII.4 Probe (RING2-TP):
(SEQ ID NO: 7)
FAM-TGG GAG GGC GAT CGC AAT CT-TAMARA

Cycling program:

10 min at 42° C. (RT step)

10 min at 95° C.

45 cycles of:
15 s at 95° C.
60 s at 60° C.

Collect fluorescence data during the “60 s at 60° C.” step.

### REFERENCES

1. Payne D C, Vinjé J, Szilagyi P G, Edwards K M, Staat M A, Weinberg G A, Hall C B, Chappell J, Bernstein D I, Curns A T, Wikswo M, Shirley S H, Hall A J, Lopman B, Parashar U D. 2013. Norovirus and Medically Attended Gastroenteritis in U.S. Children. N Engl J Med 368: 1121-1130.

2. Koo H L, Neill F H, Estes M K, Munoz F M, Cameron A, Dupont H L, Atmar R L. 2013. Noroviruses: The Most Common Pediatric Viral Enteric Pathogen at a Large University Hospital After Introduction of Rotavirus Vaccination. J Pediatr Infect Dis Soc 2: 57-60.

3. Patel M M. 2008. Systematic Literature Review of Role of Noroviruses in Sporadic Gastroenteritis. Emerg Infect Dis 14: 1224-1231.

4. Duizer E, Schwab K J, Neill F H, Atmar R L, Koopmans M P G, Estes M K. 2004. Laboratory efforts to cultivate noroviruses. J Gen Virol 85: 79-87.

5. Straub T, Honer zu B, Orosz-Coghlan P, Dohnalkova A, Mayer B, Bartholomew R, Valdez C, Bruckner-Lea C, Gerba C, Abbaszadegan M, Nickerson C. 2007. In vitro cell culture infectivity assay for human noroviruses. Emerg Infect Dis 13: 396-403.

6. Herbst-Kralovetz M M, Radtke A L, Lay M K, Hjelm B E, Bolick A N, Sarker S S, Atmar R L, Kingsley D H, Arntzen C J, Estes M K, Nickerson C A. 2013. Lack of norovirus replication and histo-blood group antigen expression in 3-dimensional intestinal epithelial cells. Emerg Infect Dis 19:431-438.

7. Papafragkou E, Hewitt J, Park G W, Greening G, Vinje J. 2013. Challenges of Culturing Human Norovirus in Three-Dimensional Organoid Intestinal Cell Culture Models. PLoS ONE 8: e63485.

8. Takanashi S, Saif L J, Hughes J H, Meulia T, Jung K, Scheuer K A, Wang Q. 2013. Failure of propagation of human norovirus in intestinal epithelial cells with microvilli grown in three-dimensional cultures. Arch Virol 159: 257-266.

9. Gonzalez-Hernandez M B, Liu T, Blanco L P, Auble H, Payne H C, Wobus C E. 2013. Murine Norovirus Transcytosis across an In Vitro Polarized Murine Intestinal Epithelial Monolayer Is Mediated by M-Like Cells. J Virol 87: 12685-12693.

10. Marionneau S, Ruvoen N, Le Moullac-Vaidye B, Clement M, Cailleau-Thomas A, Ruiz-Palacois G, Huang P, Jiang X, Le Pendu J. 2002. Norwalk virus binds to histo-blood group antigens present on gastroduodenal epithelial cells of secretor individuals. Gastroenterology 122: 1967-1977.

11. Cheetham S, Souza M, Meulia T, Grimes S, Han M G, Saif L I 2006. Pathogenesis of a Genogroup II Human Norovirus in Gnotobiotic Pigs. J Virol 80: 10372-10381.

12. Souza M, Azevedo M S P, Jung K, Cheetham S, Saif L J. 2008. Pathogenesis and Immune

Responses in Gnotobiotic Calves after Infection with the Genogroup II.4-H566 Strain of Human Norovirus. J Virol 82: 1777-1786.

13. Mumphrey S M, Changotra H, Moore T N, Heimann-Nichols E R, Wobus C E, Reilly M J, Moghadamfalahi M, Shukla D, Karst S M. 2007. Murine Norovirus 1 Infection Is Associated with Histopathological Changes in Immunocompetent Hosts, but Clinical Disease Is Prevented by STAT1-Dependent Interferon Responses. J Virol 81: 3251-3263.

14. Wobus C E, Karst S M, Thackray L B, Chang K-O, Sosnovtsev S V, Belliot G, Krug A, Mackenzie J M, Green K Y, Virgin H W. 2004. Replication of Norovirus in Cell Culture Reveals a Tropism for Dendritic Cells and Macrophages. PLoS Biol 2:e432.

15. Lay M K, Atmar R L, Guix S, Bharadwaj U, He H, Neill F H, Sastry K J, Yao Q, Estes M K. 2010. Norwalk virus does not replicate in human macrophages or dendritic cells derived from the peripheral blood of susceptible humans. Virology 406: 1-11.

16. Chan M C-W, Ho W-S, Sung J J-Y. 2011. In Vitro Whole-Virus Binding of a Norovirus Genogroup II Genotype 4 Strain to Cells of the Lamina Propria and Brunner's Glands in the Human Duodenum. J Virol 85: 8427-8430.

17. Bok K, Parra G I, Mitra T, Abente E, Shaver C K, Boon D, Engle R, Yu C, Kapikian A Z, Sosnovtsev S V, Purcell R H, Green K Y. 2011. Chimpanzees as an animal model for human norovirus infection and vaccine development. Proc Natl Acad Sci 108: 325-330.

18. Taube S, Kolawole A O, Hohne M, Wilkinson J E, Handley S A, Perry J W, Thackray L B, Akkina R, Wobus C E. 2013. A Mouse Model for Human Norovirus. mBio 4:e00450-13.

19. Zhu S, Regev D, Watanabe M, Hickman D, Moussatche N, Jesus D M, Kahan S M, Napthine S, Brierley I, Hunter R N, Devabhaktuni D, Jones M K, Karst S M. 2013. Identification of Immune and Viral Correlates of Norovirus Protective Immunity through Comparative Study of Intra-Cluster Norovirus Strains. PLoS Pathog 9:e1003592.

20. Miura T, Sano D, Suenaga A, Yoshimura T, Fuzawa M, Nakagomi T, Nakagomi O, Okabe S. 2013. Histo-Blood Group Antigen-Like Substances of Human Enteric Bacteria as Specific Adsorbents for Human Noroviruses. J Virol 87: 9441-51.

21. Tan M, Jiang X. 2005. Norovirus and its histo-blood group antigen receptors: an answer to a historical puzzle. Trends Microbiol 13: 285-293.

22. Andersson M, Carlin N, Leontein K, Lindquist U, Slettengren K. 1989. Structural studies of the O-antigenic polysaccharide of Escherichia coli O86, which possesses blood-group B activity. Carbohydr Res 185: 211-223.

23. Aspinall G O, Monteiro M A. 1996. Lipopolysaccharides of Helicobacter pylori Strains P466 and MO19: Structures of the O Antigen and Core Oligosaccharide Regions†. Biochemistry (Mosc) 35: 2498-2504.

24. Rasko D A, Wang G, Monteiro M A, Palcic M M. 2000. Synthesis of mono- and di-fucosylated type I Lewis blood group antigens by Helicobacter pylori. Eur J Biochem 267: 6059-6066.

25. Yi W, Shao J, Zhu L, Li M, Singh M, Lu Y, Lin S, Li H, Ryu K, Shen J, Guo H, Yao Q, Bush C A, Wang P G. 2005. Escherichia coli 086 0-Antigen Biosynthetic Gene Cluster and Stepwise Enzymatic Synthesis of Human Blood Group B Antigen Tetrasaccharide. J Am Chem Soc 127: 2040-2041.

26. Kim K J, Kanellopoulos-Langevin C, Merwin R M, Sachs D H, Asofsky R. 1979. Establishment and Characterization of BALB/c Lymphoma Lines with B Cell Properties. J Immunol 122: 549-554.

27. Lanier L L, Lynes M, Haughton G, Wettstein P J. 1978. Novel type of murine B-cell lymphoma. Nature 271: 554-555.

28. Klein G, Lindahl T, Jondal M, Leibold W, Menezes J, Nilsson K, Sundstrom C. 1974. Continuous Lymphoid Cell Lines with Characteristics of B Cells (Bone-Marrow-Derived), Lacking the Epstein-Barr Virus Genome and Derived from Three Human Lymphomas. Proc Natl Acad Sci U S A 71: 3283-3286.

29. Thackray L B, Wobus CeE, Chachu K A, Liu B, Alegre E R, Henderson K S, Kelley S T, Virgin H W. 2007. Murine Noroviruses Comprising a Single Genogroup Exhibit Biological Diversity despite Limited Sequence Divergence. J Virol 81: 10460-10473.

30. Park Y, Cho Y-H, Ko G. 2011. A duplex real-time RT-PCR assay for the simultaneous genogroup-specific detection of noroviruses in both clinical and environmental specimens. Virus Genes.

31. Changotra H, Jia Y, Moore T N, Liu G, Kahan S M, Sosnovtsev S V, Karst S M. 2009. Type I and Type II Interferons Inhibit the Translation of Murine Norovirus Proteins. J Virol 83: 5683-5692.

32. Karst S M, Wobus C E, Lay M, Davidson J, Virgin H W. 2003. STAT1-Dependent Innate Immunity to a Norwalk-Like Virus. Science 299: 1575-1578.

33. Kahan S M, Liu G, Reinhard M K, Hsu C C, Livingston R S, Karst S M. 2011. Comparative murine norovirus studies reveal a lack of correlation between intestinal virus titers and enteric pathology. Virology 421: 202-210.

34. Rakoff-Nahoum S, Paglino J, Eslami-Varzaneh F, Edberg S, Medzhitov R. 2004. Recognition of Commensal Microflora by Toll-Like Receptors Is Required for Intestinal Homeostasis. Cell 118: 229-241.

35. Kuss S K, Best G T, Etheredge C A, Pruijssers A J, Frierson J M, Hooper L V, Dermody T S, Pfeiffer J K. 2011. Intestinal Microbiota Promote Enteric Virus Replication and Systemic Pathogenesis. Science 334: 249-252.

36. Clayson E T, Brando L V, Compans R W. 1989. Release of simian virus 40 virions from epithelial cells is polarized and occurs without cell lysis. J Virol 63: 2278-2288.

37. Jourdan N, Maurice M, Delautier D, Quero A M, Servin A L, Trugnan G. 1997. Rotavirus is released from the apical surface of cultured human intestinal cells through nonconventional vesicular transport that bypasses the Golgi apparatus. J Virol 71: 8268-8278.

38. Roussarie J-P, Rufflé C, Edgar J M, Griffiths I, Brahic M. 2007. Axon Myelin Transfer of a Non-Enveloped Virus. PLoS ONE 2:e1331.

39. Taylor M P, Burgon T B, Kirkegaard K, Jackson W T. 2009. Role of Microtubules in Extracellular Release of Poliovirus. J Virol 83: 6599-6609.

40. Lai C M, Mainou B A, Kim K S, Dermody T S. 2013. Directional Release of Reovirus from the Apical Surface of Polarized Endothelial Cells. mBio 4.

41. Feng Z, Hensley L, McKnight K L, Hu F, Madden V, Ping L, Jeong S-H, Walker C, Lanford R E, Lemon S M. 2013. A pathogenic picornavirus acquires an envelope by hijacking cellular membranes. Nature 496: 367-371.

42. Wesemann D R, Portuguese A J, Meyers R M, Gallagher M P, Cluff-Jones K, Magee J M, Panchakshari R A, Rodig S J, Kepler T B, Alt F W. 2013. Microbial colonization influences early B-lineage development in the gut lamina propria. Nature 501: 112-115.

43. Zhang X, Zheng Z, Shu B, Liu X, Zhang Z, Liu Y, Bai B, Hu Q, Mao P, Wang H. 2013. Human Astrocytic Cells Support Persistent Coxsackievirus B3 Infection. J Virol 87: 12407-12421.

44. McFadden N, Bailey D, Carrara G, Benson A, Chaudhry Y, Shortland A, Heeney J, Yarovinsky F, Simmonds P, Macdonald A, Goodfellow I. 2011. Norovirus Regulation of the Innate Immune Response and Apoptosis Occurs via the Product of the Alternative Open Reading Frame 4. PLoS Pathog 7:e1002413.

45. Hsu C C, Riley L K, Wills H M, Livingston R S. 2006. Persistent Infection with and Serologic Crossreactivity of Three Novel Murine Noroviruses. Comp Med 56: 247-251.

46. Arias A, Bailey D, Chaudhry Y, Goodfellow I G. 2012. Development of a Reverse Genetics System for Murine Norovirus 3; Long-Term Persistence Occurs in the Caecum and Colon. J Gen Virol 93: 1432-1441.

47. Patterson T, Hutchings P, Palmer S. 1993. Outbreak of SRSV gastroenteritis at an international conference traced to food handled by a post-symptomatic caterer. Epidemiol Infect 111: 157-162.

48. Rockx B, de Wit M, Vennema H, Vinjé J, de Bruin E, van Duynhoven Y, Koopmans M. 2002. Natural History of Human Calicivirus Infection: A Prospective Cohort Study. Clin Infect Dis 35: 246-253.

49. Atmar R L, Opekun A R, Gilger M A, Estes M K, Crawford S E, Neill F H, Graham D Y. 2008. Norwalk virus shedding after experimental human infection. Emerg Infect Dis 14: 1553-1557.

50. Karst S M, Wobus C E, Goodfellow I G, Green K Y, Virgin H W. 2014. Advances in Norovirus Biology. Cell Host Microbe 15: 668-680.

51. Tamura M, Natori K, Kobayashi M, Miyamura T, Takeda N. 2000. Interaction of Recombinant Norwalk Virus Particles with the 105-Kilodalton Cellular Binding Protein, a Candidate Receptor Molecule for Virus Attachment. J Virol 74: 11589-11597.

52. White L J, Ball J M, Hardy M E, Tanaka T N, Kitamoto N, Estes M K. 1996. Attachment and entry of recombinant Norwalk virus capsids to cultured human and animal cell lines. J Virol 70: 6589-6597.

53. Bomsel M. 1997. Transcytosis of infectious human immunodeficiency virus across a tight human epithelial cell line barrier. Nat Med 3: 42-47.

54. Di Pasquale G, Chiorini J A. 2006. AAV Transcytosis through Barrier Epithelia and Endothelium. Mol Ther 13: 506-516.

55. Dotzauer A, Brenner M, Gebhardt U, Vallbracht A. 2005. IgA-coated particles of Hepatitis A virus are translocalized antivectorially from the apical to the basolateral site of polarized epithelial cells via the polymeric immunoglobulin receptor. J Gen Virol 86: 2747-2751.

56. Martin-Latil S, Gnadig N F, Mallet A, Desdouits M, Guivel-Benhassine F, Jeannin P, Prevost M-C, Schwartz O, Gessain A, Ozden S, Ceccaldi P-E. 2012. Transcytosis of HTLV-1 across a tight human epithelial barrier and infection of subepithelial dendritic cells. Blood 120: 572-580.

57. Ouzilou L, Caliot E, Pelletier I, Prévost M-C, Pringault E, Colbére-Garapin F. 2002. Poliovirus transcytosis through M-like cells. J Gen Virol 83: 2177-2182.

58. Tugizov S M, Herrera R, Palefsky J M. 2013. Epstein-Barr Virus Transcytosis through Polarized Oral Epithelial Cells. J Virol 87: 8179-8194.

59. Huang P, Farkas T, Marionneau S, Zhong W, Ruvoën-Clouet N, Morrow AL, Altaye M, Pickering L K, Newburg D S, LePendu J, Jiang X. 2003. Noroviruses Bind to Human ABO, Lewis, and Secretor Histo-Blood Group Antigens: Identification of 4 Distinct Strain-Specific Patterns. J Infect Dis 188: 19-31.

60. Springer G F, Williamson P, Brandes W C. 1961. Blood Group Activity of Gram-Negative Bacteria. J Exp Med 113: 1077-1093.

61. Robinson C M, Jesudhasan P R, Pfeiffer J K. 2014. Bacterial Lipopolysaccharide Binding Enhances Virion Stability and Promotes Environmental Fitness of an Enteric Virus. Cell Host Microbe 15: 36-46.

62. Kuss S K, Best G T, Etheredge C A, Pruijssers A J, Frierson J M, Hooper L V, Dermody T S, Pfeiffer J K. 2011. Intestinal Microbiota Promote Enteric Virus Replication and Systemic Pathogenesis. Science 334: 249-252.

63. Kane M, Case L K, Kopaskie K, Kozlova A, MacDearmid C, Chervonsky A V, Golovkina T V. 2011. Successful Transmission of a Retrovirus Depends on the Commensal Microbiota. Science 334: 245-249.

64. Uchiyama R, Chassaing B, Zhang B, Gewirtz A T. 2014. Antibiotic Treatment Suppresses Rotavirus Infection and Enhances Specific Humoral Immunity. J Infect Dis 210: 171-182.

65. Karst S M, Wobus C E. 2015. A Working Model of How Noroviruses Infect the Intestine. PLoS Pathog 11:e1004626.

1-42. (canceled)

43. A method for continuous culture of norovirus, comprising:
a) contacting norovirus in an in vitro cell culture system comprising intestinal epithelial cells grown on a transwell membrane, wherein the basal side comprises B-cells or contacting a norovirus containing sample to B-cells or macrophages;
b) allowing the norovirus to replicate; and
c) passaging the norovirus produced in the culture vessel to a subsequent in vitro cell culture vessel comprising intestinal epithelial cells grown on a transwell membrane, wherein the basal side comprises B-cells or passaging the norovirus produced in the culture vessel to a subsequent in vitro cell culture vessel containing B-cells or macrophages.

44. The method of claim 43, comprising repeating the steps.

45. The method of claim 43, further comprising adding HBGA-like carbohydrates or one or more sources of HBGA-like carbohydrates.

46. The method of claim 45, wherein in the HBGA-like carbohydrates are from a bacteria.

47. The method of claim 45, wherein the HBGA-like carbohydrates are isolated HBGA-like carbohydrates.

48. A diagnostic method for norovirus detection, comprising:
a) obtaining a biological or diagnostic sample and contacting said sample with an in vitro cell culture system comprising B-cells, or to an in vitro cell culture vessel comprising a transwell cell culture system comprising intestinal epithelial cells grown on the transwell membrane and B-cells in the lower (basal) chamber, wherein the B-cells are located on the basal side of the intestinal epithelial cells; and
b) after a predetermined time. testing the supernatant of the B-cells for norovirus attachment or replication.

49. The method of claim 48, further comprising identifying the norovirus found.

50. The method of claim 48, wherein the biological or diagnostic sample is a food sample, food additive, or other agent required for the processing or production of a food product.

51. A screening test for an active agent for inhibiting norovirus replication or infection, comprising:
a) contacting a candidate active agent with an in vitro cell culture system comprising B-cells, or to an in vitro cell culture vessel comprising a transwell cell culture system comprising intestinal epithelial cells grown on the transwell membrane and B-cells in the lower chamber, wherein the B-cells are located on the basal side of the intestinal epithelial cells infected with norovirus;
b) allowing the norovirus to attach or replicate; and
c) detecting the inhibition of virus attachment or inhibition of virus replication in the supernatant or the B-cells in the culture system for the presence of norovirus.
